(19) United States
(12) Patent Application Publication
(10) Pub. No.: US 2025/0262209 A1
(43) Pub. Date: Aug. 21, 2025

(54) TREATMENT OF GASTROINTESTINAL DISEASES

(71) Applicant: Aclipse Two, Inc., Radnor, PA (US)

(72) Inventor: Ning Shan, Chandler, AZ (US)

(21) Appl. No.: 18/857,415

(22) PCT Filed: Apr. 19, 2023
(86) PCT No.: PCT/US2023/065941

§ 371 Date: Oct. 16, 2024

Related U.S. Application Data
(60) Provisional application No. 63/332,778, filed on Apr. 20, 2022.

(51) Int. Cl. A61K 31/506; A61P 1/00

(52) U.S. Cl. CPC A61K 31/506; A61P 1/00

(57) ABSTRACT

Disclosed herein are the use of thiazolidinediones for the treatment of digestive diseases including gastroparesis.

### CROSS-REFERENCE TO RELATED APPLICATIONS

The present application claims the benefit of U.S. Provisional Patent Application 63/332,778 filed Apr. 20, 2022, the entire contents of which are incorporated by reference herein.

### FIELD

The present disclosure relates to a therapeutic agent and methods for the treatment of gastrointestinal disorders such as gastroparesis.

### BACKGROUND

Digestive diseases are disorders of the digestive tract, which is also called the gastrointestinal (GI) tract. GI diseases and illnesses are any ailments linked to the GI system, including the entire GI tract, from the mouth to the anus, including the esophagus, stomach, and intestines. Diagnoses may include acute, short-term illnesses. GI diseases may also include more chronic diagnoses and may require long-term, specialty treatment.

Conditions range from mild to serious. Some common problems include heartburn, cancer, irritable bowel syndrome (IBS), and lactose intolerance. Other digestive diseases include gallstones, cholecystitis, cholangitis, rectal diseases (e.g., anal fissure, hemorrhoids, proctitis, and rectal prolapse), esophagus diseases (e.g., stricture and achalasia and esophagitis), stomach diseases (e.g., gastritis, gastric ulcers usually caused by Helicobacter pylori infection and cancer), liver diseases (e.g., hepatitis B or hepatitis C, cirrhosis, liver failure, and autoimmune and alcoholic hepatitis), pancreatitis and pancreatic pseudocyst, intestinal diseases (e.g., polyps and cancer, infections, celiac disease, Crohn disease, ulcerative colitis, diverticulitis, malabsorption, short bowel syndrome, and intestinal ischemia), gastroesophageal reflux disease (GERD), peptic ulcer disease, and hiatal hernia.

Among all digestive diseases, gastroparesis is a disorder characterized by delayed gastric emptying (DGE) in the absence of mechanical obstruction. Symptoms are chronic with episodic exacerbation. The idiopathic form of the disorder, which accounts for the greatest number of cases, predominantly affects young adult females. Gastroparesis is also frequently associated with diabetes (diabetic gastroparesis), which likely occurs because of impaired neural control of gastric motility. In addition, acute hyperglycemia has the potential to slow gastric emptying and decrease the effects of prokinetic drugs.

On the molecular level, gastroparesis can be caused by the loss of neuronal nitric oxide expression since the cells in the GI tract secrete nitric oxide. This important signaling molecule has various responsibilities in the GI tract and in muscles throughout the body. When nitric oxide levels are low, the smooth muscle and other organs may not be able to function properly. Other important components of the stomach are the interstitial cells of Cajal (ICC) which act as a pacemaker since they transduce signals from motor neurons to produce an electrical rhythm in the smooth muscle cells. Lower nitric oxide levels also correlate with loss of ICC cells, which can ultimately lead to the loss of function in the smooth muscle in the stomach, as well as in other areas of the GI tract. Pathogenesis of symptoms in diabetic gastroparesis include (1) loss of gastric neurons containing nitric oxide synthase (NOS) which is responsible for defective accommodation reflex, and leads to early satiety and postprandial fullness; (2) impaired electromechanical activity in the myenteric plexus which is responsible for delayed gastric emptying, resulting in nausea and vomiting; (3) sensory neuropathy in the gastric wall which may be responsible for epigastric pain; and (4) abnormal pacemaker activity (tachybradyarrhythmia) which may generate a noxious signal transmitted to the CNS to evoke nausea and vomiting.

Macrophages also play a role in the development and progression of gastroparesis. In particular, macrophages infiltrate the smooth muscle layer of the stomach in people with gastroparesis and release pro-inflammatory molecules that contribute to the damage and dysfunction of the stomach muscles. In cases of diabetic gastroparesis, high blood sugar levels activate macrophages, leading to inflammation and damage to the nerves and muscles of the stomach. There are two different subtypes of macrophages that have different functions in the immune system, i.e., M1 and M2 macrophages. M1 macrophages are involved in the inflammatory response and are activated in response to pro-inflammatory signals. They are important in fighting infections. M1 macrophages produce pro-inflammatory cytokines and reactive oxygen species, which help to kill invading pathogens and promote the recruitment of other immune cells to the site of infection. On the other hand, M2 macrophages are involved in tissue repair and immune regulation, and are activated in response to anti-inflammatory signals. They are important in resolving inflammation and promoting tissue remodeling and healing. M2 macrophages produce anti-inflammatory cytokines and growth factors that help to repair damaged tissues and promote angiogenesis. Overall, M1 and M2 macrophages represent two different functional states of macrophages that allow them to respond to different signals and perform different roles in the immune system. The balance between M1 and M2 macrophages is important in maintaining immune homeostasis and promoting proper immune responses. Drugs that can inhibit M1 macrophage, promote M2 macrophage and/or enable M1-to-M2 macrophage repolarization may be able to treat digestive diseases including gastroparesis.

Because the signs and symptoms of gastroparesis overlap with other GI conditions, gastroparesis is sometimes incorrectly diagnosed as bowel obstruction, functional dyspepsia, irritable bowel syndrome, or peptic ulcer disease. In a patient with signs and symptoms suggestive of gastroparesis, a finding of DGE in the absence of an obstruction or alternative diagnosis provides critical support for the diagnosis of gastroparesis and can be assessed using either gastric emptying scintigraphy, the gastric emptying breath test, or the SmartPill™ motility testing system.

Approved medications to treat gastroparesis can be categorized into two different groups. The first group of medications only provides temporary relief to some disease symptoms such as nausea and vomiting including diphenhydramine and ondansetron, and prochlorperazine. The second group of medications is to stimulate the stomach muscles and includes metoclopramide (e.g., Reglan®) and erythromycin. However, both metoclopramide and erythromycin have risks of significant side effects. Clearly, there is an urgent medical need for development of safe and effective therapies to treat patients with gastroparesis.

Thiazolidinediones represents a novel therapeutic strategy that could slow, halt, or reverse the underlying disease process in diseases involving digestive diseases, such as gastroparesis.

### SUMMARY

Disclosed herein are methods of treating GI diseases, comprising administering to a subject in need thereof an effective amount of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone. In some embodiments, the thiazolidinedione is lobeglitazone. In some embodiments, the GI disease is gastroparesis. In some embodiments, the gastroparesis is idiopathic gastroparesis, diabetic gastroparesis, post-surgical gastroparesis, or medication-induced gastroparesis.

Lobeglitazone is a thiazolidinediones with a chemical name of 5-(4-(2-((6-(4-Methoxyphenoxy)pyrimidin-4-yl)(methyl)amino) ethoxy)benzyl)thiazolidine-2,4-dione. Lobeglitazone was approved as an antidiabetic drug under the brand name Duvie®. As an agonist for both PPARα and PPARγ, it works as an insulin sensitizer by binding to the PPAR receptors in fat cells and making the cells more responsive to insulin. Lobeglitazone showed much higher potency in PPARγ activity than pioglitazone. Lobeglitazone is depicted by the following chemical structure:

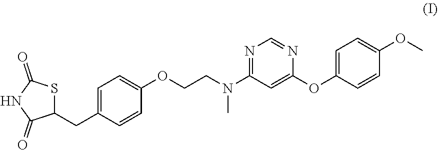 [SMILES: COc1ccc(Oc2cc(N(C)CCOc3ccc(CC4SC(=O)NC4=O)cc3)ncn2)cc1]

Gastroparesis is a disorder characterized by delayed gastric emptying (DGE) in the absence of mechanical obstruction. Symptoms are chronic with episodic exacerbation. The idiopathic form of the disorder, which accounts for the greatest number of cases, predominantly affects young adult females. Gastroparesis is also frequently associated with diabetes (diabetic gastroparesis), which likely occurs because of impaired neural control of gastric motility. Macrophages play a role in the development and progression of gastroparesis.

In some embodiments, the GI disease affects a digestive organ or tissue selected from mouth, pharynx (throat), esophagus, stomach, small intestine, large intestine, rectum, anus, salivary glands, liver, gallbladder, and pancreas.

In some embodiments, method comprises treating a GI system cell selected from an absorptive cell (enterocyte), a goblet cell, a pancreatic islet cell, an enteroendocrine cell, a hepatocyte, a paneth cell, a fenestrated hepatic endothelial cell, a kupffer cell, a serous cell, a gastric chief cell, a mucous cell, a smooth muscle cell, a gastric parietal cell, a myoepithelial cell, a stem cell, a gastric surface mucous cell, a pancreatic acinar cell, a taste bud, a type of interstitial cells of Cajal (ICC), and a neuronal cell.

In some embodiments, the cell is an animal cell. In some embodiments, the cell in a human cell. In some embodiments, the cell is treated in vitro. In some embodiments, the cell is treated ex vivo. In some embodiments, the cell is treated in vivo.

In some embodiments, the cell is in, or from, a subject having a GI disease or disorder or is at risk of the GI disease or disorder. selected from any one or more of abdominal adhesions, acid reflux (gastroesophageal reflux disease or GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, inflammatory bowel disease (IBD), irritable bowel syndrome (IBS), lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, Zollinger-Ellison syndrome, or a condition associated therewith. In some embodiments, the disease or disorder is gastroparesis. In some embodiments, the gastroparesis is idiopathic gastroparesis, diabetic gastroparesis, post-surgical gastroparesis, or medication-induced gastroparesis.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to relieve one or more signs and/or symptoms of gastroparesis. In some embodiments, the thiazolidinedione is lobeglitazone. In some embodiments, the signs and symptoms of gastroparesis are measured by the ANMS GCSI-DD. In some embodiments, the signs and symptoms of gastroparesis are measured by the change of GCSI-DD. In some embodiments, the signs and symptoms of gastroparesis are measured by the change of GCSI-DD score from baseline to 4-week treatment. In some embodiments, the signs and symptoms of gastroparesis are measured by the change of GCSI-DD score from baseline to 8-week treatment. In some embodiments, the signs and symptoms of gastroparesis are measured by the change of GCSI-DD score from baseline to 12-week treatment. In some embodiments, the signs and symptoms of gastroparesis are measured by the change of GCSI-DD score from baseline to 1-year treatment. In some embodiments, the signs and symptoms of gastroparesis are measured by the change of GCSI-DD score from baseline to more than 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the severity of nausea in a subject with gastroparesis. In some embodiments, the thiazolidinedione is lobeglitazone. In some embodiments, the decrease the severity of nausea is measured by the change of severity of nausea from baseline to 4-week treatment. In some embodiments, the decrease the severity of nausea is measured by the change of severity of nausea from baseline to 8-week treatment. In some embodiments, the decrease the severity of nausea is measured by the change of severity of nausea from baseline to 12-week treatment. In some embodiments, the decrease the severity of nausea is measured by the change of severity of nausea from baseline to 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the severity of early satiety in a subject with gastroparesis. In some embodiments, the thiazolidinedione is lobeglitazone. In some embodiments, the decrease the severity of early satiety is measured by the change of severity of early satiety from baseline to 4-week treatment. In some embodiments, the decrease the severity of early satiety is measured by the change of severity of early satiety from baseline to 8-week treatment. In some embodiments, the decrease the severity of early satiety is measured by the change of severity of early satiety from baseline to 12-week treatment. In some embodiments, the decrease the severity of early satiety is measured by the change of severity of early satiety from baseline to 1-year treatment. In some embodiments, the decrease the severity of early satiety is measured by the change of severity of early satiety from baseline to more than 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the severity of postprandial fullness in a subject with gastroparesis. In some embodiments, the thiazolidinedione is lobeglitazone. In some embodiments, the decrease the severity of postprandial fullness is measured by the change of severity of postprandial fullness from baseline to 4-week treatment. In some embodiments, the decrease the severity of postprandial fullness is measured by the change of severity of postprandial fullness from baseline to 8-week treatment. In some embodiments, the decrease the severity of postprandial fullness is measured by the change of severity of postprandial fullness from baseline to 12-week treatment. In some embodiments, the decrease the severity of postprandial fullness is measured by the change of severity of postprandial fullness from baseline to 1-year treatment. In some embodiments, the decrease the severity of postprandial fullness is measured by the change of severity of postprandial fullness from baseline to more than 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the severity of upper abdominal pain in a subject with gastroparesis. In some embodiments, the thiazolidinedione is lobeglitazone. In some embodiments, the decrease the severity of upper abdominal pain is measured by the change of severity of upper abdominal pain from baseline to 4-week treatment. In some embodiments, the decrease the severity of upper abdominal pain is measured by the change of severity of upper abdominal pain from baseline to 8-week treatment. In some embodiments, the decrease the severity of upper abdominal pain is measured by the change of severity of upper abdominal pain from baseline to 12-week treatment. In some embodiments, the decrease the severity of upper abdominal pain is measured by the change of severity of upper abdominal pain from baseline to 1-year treatment. In some embodiments, the decrease the severity of upper abdominal pain is measured by the change of severity of upper abdominal pain from baseline to more than 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the frequency of vomiting episodes in a subject with gastroparesis. In some embodiments, the thiazolidinedione is lobeglitazone. In some embodiments, the decrease the severity of vomiting episodes is measured by the change of severity of vomiting episodes from baseline to 4-week treatment. In some embodiments, the decrease the severity of vomiting episodes is measured by the change of severity of vomiting episodes from baseline to 8-week treatment. In some embodiments, the decrease the severity of vomiting episodes is measured by the change of severity of vomiting episodes from baseline to 12-week treatment. In some embodiments, the decrease the severity of vomiting episodes is measured by the change of severity of vomiting episodes from baseline to 1-year treatment. In some embodiments, the decrease the severity of vomiting episodes is measured by the change of severity of vomiting episodes from baseline to more than 1-year treatment.

Also disclosed herein is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to decrease the overall severity of gastroparesis. In some embodiments, the thiazolidinedione is lobeglitazone. In some embodiments, the decrease the severity of overall severity of gastroparesis is measured by the change of severity of overall severity of gastroparesis from baseline to 4-week treatment. In some embodiments, the decrease the severity of overall severity of gastroparesis is measured by the change of severity of overall severity of gastroparesis from baseline to 8-week treatment. In some embodiments, the decrease the severity of overall severity of gastroparesis is measured by the change of severity of overall severity of gastroparesis from baseline to 12-week treatment. In some embodiments, the decrease the severity of overall severity of gastroparesis is measured by the change of severity of overall severity of gastroparesis from baseline to 1-year treatment. In some embodiments, the decrease the severity of overall severity of gastroparesis is measured by the change of severity of overall severity of gastroparesis from baseline to more than 1-year treatment.

Also disclosed here is the use of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone, to slow down, stop, or reverse disease progression of a GI disease. In some embodiments, the thiazolidinedione is lobeglitazone.

Also disclosed herein are methods of treating a GI disease in a mammal comprising administering an effective amount of a thiazolidinedione selected from one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, and balaglitazone to the mammal. In some embodiments, the GI disease is gastroparesis or IBD. In some embodiments, the thiazolidinedione is lobeglitazone.

In some embodiments, gastroparesis is idiopathic gastroparesis, diabetic gastroparesis, post-surgical gastroparesis, or medication-induced gastroparesis.

Also disclosed herein are methods of inhibiting M1 macrophage differentiation in an individual, comprising administering an effective amount of lobeglitazone to the individual. In some embodiments, as a result of the inhibition of M1 macrophage differentiation, a GI system disorder is treated to the individual.

Also disclosed herein are methods of promoting M1 macrophage differentiation in an individual, comprising administering an effective amount of pioglitazone or rosiglitazone to the individual.

Also disclosed herein are methods of promoting M2 macrophage differentiation in an individual, comprising administering an effective amount of lobeglitazone, pioglitazone, or rosiglitazone to the individual. In some embodiments, as a result of the promotion of M2 macrophage differentiation, a GI system disorder is treated to the individual. Also disclosed herein are methods of promoting M1-to-M2 macrophage repolarization in an individual, comprising administering an effective amount of lobeglitazone, pioglitazone or rosiglitazone to the individual. In some embodiments, as a result of the promotion of M1-to-M2 macrophage repolarization, a GI system disorder is treated to the individual. In some embodiments, the thiazolidinedione is lobeglitazone. In some embodiments, the GI system disorder is selected from abdominal adhesions, acid reflux (GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, IBD, IBS, lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, and Zollinger-Ellison syndrome, or a condition associated therewith.

### BRIEF DESCRIPTION OF THE DRAWINGS

FIG. 1 depicts the experimental schedule of lobeglitazone study in a mouse model of inflammatory bowel disease (IBD).

FIG. 2 depicts the animal body weights of naïve group, 5% dextran sodium sulfate (DSS) model group (Model), lobeglitazone groups, and cyclosporine A group (mpk denotes mg/kg). * p<0.05, ** p<0.01 vs. Model control group.

FIG. 3 depicts the change in animal body weight of naïve group, 5% DSS model group, lobeglitazone groups, and cyclosporine A group. * p<0.05, ** p<0.01 vs. Model control group.

FIG. 4 depicts the disease activity index (DAI) scores of naïve group, 5% DSS model group, lobeglitazone groups, and cyclosporine A group. ** p<0.01, *** p<0.001 vs. Model control group.

FIG. 5 depicts the post-treatment colon weights of naïve group, 5% DSS model group, lobeglitazone groups, and cyclosporine A group.

FIG. 6 depicts the post-treatment colon lengths of naïve group, 5% DSS model group, lobeglitazone groups, and cyclosporine A group. * p<0.05, *** p<0.001 vs. Model control group.

FIG. 7 depicts the post-treatment colon weight/length ratio of naïve group, 5% DSS model group, lobeglitazone groups, and cyclosporine A group. * p<0.05 vs. Model control group.

FIG. 8 depicts the post-treatment TNF-α gene expression in the colon of naïve group, 5% DSS model group, lobeglitazone groups, and cyclosporine A group. * p<0.05, *** p<0.001 vs. Model control group.

FIG. 9 depicts the post-treatment IL-10 gene expression in the colon of naïve group, 5% DSS model group, lobeglitazone groups, and cyclosporine A group.

FIG. 10 depicts the post-treatment CCL2 gene expression in the colon of naïve group, 5% DSS model group, lobeglitazone groups, and cyclosporine A group. * p<0.05, ** p<0.01 vs. Model control group.

FIG. 11 depicts the post-treatment IL-1B gene expression in the colon of naïve group, 5% DSS model group, lobeglitazone groups, and cyclosporine A group. *** p<0.001 vs. Model control group.

FIG. 12 depicts the post-treatment inflammation/tissue damage score (total hematoxylin and eosin (H&E) scores) of naïve group, 5% DSS model group, lobeglitazone groups, and cyclosporine A group. ** p<0.01, ** p<0.001 vs. Model control group.

FIG. 13 depicts the post-treatment number of goblet cells (periodic acid-Schiff (PAS) staining) of naïve group, 5% DSS model group, lobeglitazone groups, and cyclosporine A group. * p<0.05 vs. Model control group.

FIG. 14 depicts M1 macrophage cytokine IL-23 expression after differentiation with lobeglitazone, pioglitazone, or rosiglitazone.

FIG. 15 depicts M1 macrophage cytokine IL-1b expression after differentiation with lobeglitazone, pioglitazone, or rosiglitazone.

FIG. 16 depicts M1-like macrophage cytokine TNF-alpha expression after differentiation with lobeglitazone, pioglitazone, or rosiglitazone.

FIG. 17 depicts M1-like macrophage cytokine IL-12p70 expression after differentiation with lobeglitazone, pioglitazone, or rosiglitazone.

FIG. 18 depicts M1-like macrophage cytokine IL-6 expression after differentiation with lobeglitazone, pioglitazone, or rosiglitazone.

FIG. 19 depicts M2 macrophage surface markers expression following treatment with lobeglitazone, pioglitazone, or rosiglitazone (donor 1).

FIG. 20 depicts M2 macrophage surface markers expression following treatment with lobeglitazone, pioglitazone, or rosiglitazone (donor 2).

FIG. 21 depicts M2 macrophage surface markers expression following treatment with lobeglitazone, pioglitazone, or rosiglitazone (donor 3).

FIG. 22 depicts M1 macrophage associated cytokines expression in cells from three donors following treatment with lobeglitazone, pioglitazone, or rosiglitazone.

FIG. 23 depicts M2 macrophage associated cytokines expression in cells from three donors following treatment with lobeglitazone, pioglitazone, or rosiglitazone.

FIG. 24 depicts macrophage-derived chemokine (MDC) expression following treatment in cells from three donors following treatment with lobeglitazone, pioglitazone, or rosiglitazone.

FIG. 25 depicts Eotaxin-2 expression following treatment in cells from three donors following treatment with lobeglitazone, pioglitazone, or rosiglitazone.

FIG. 26 depicts IL-1RA expression following treatment in cells from three donors following treatment with lobeglitazone, pioglitazone, or rosiglitazone.

FIG. 27 depicts M2 macrophage surface marker expression following treatment with lobeglitazone, pioglitazone, or rosiglitazone (donor 1).

FIG. 28 depicts M1 macrophage associated cytokine expression following treatment with lobeglitazone, pioglitazone, or rosiglitazone.

FIG. 29 depicts M2 macrophage associated cytokine expression following treatment with lobeglitazone, pioglitazone, or rosiglitazone.

FIG. 30 depicts MDC expression following treatment with lobeglitazone, pioglitazone, or rosiglitazone.

FIG. 31 depicts Eotaxin-2 expression following treatment with lobeglitazone, pioglitazone, or rosiglitazone.

FIG. 32. depicts IL-1RA expression following treatment with lobeglitazone, pioglitazone, or rosiglitazone.

### DETAILED DESCRIPTION

Disclosed herein are methods of treating a GI disease or disorder disclosed herein with a thiazolidinedione.

The term “thiazolidinedione” refers to a class of heterocyclic glitazones compounds which comprise a five-membered C3NS ring, including prodrugs, salts, solvates, hydrates, cocrystals, enantiomers, and deuterated forms thereof. As used herein, a thiazolidinedione includes, but is not limited to, one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, balaglitazone, and other thiazolidinedione molecules. In some embodiments disclosed herein, the methods include using any thiazolidinedione. In some embodiments, the thiazolidinedione is lobeglitazone. In some embodiments, the methods specifically exclude use of one or more thiazolidinedione disclosed herein. In some embodiments, the thiazolidinedione is not pioglitazone. In some embodiments, the thiazolidinedione is not pioglitazone or rosiglitazone.

Lobeglitazone (Duvie®, Chong Kun Dang) is a thiazolidinedione with a chemical name of 5-(4-(2-((6-(4-Methoxyphenoxy)pyrimidin-4-yl)(methyl)amino) ethoxy)benzyl) thiazolidine-2,4-dione. As an agonist for both peroxisome proliferator-activated receptor-alpha (PPARα) and gamma (PPARγ), lobeglitazone works as an insulin sensitizer by binding to the PPAR receptors in fat cells and making the cells more responsive to insulin.

Pioglitazone (Actos®, Takeda) is a thiazolidinedione with a chemical name (±)-5-[p-[2-(5-Ethyl-2-pyridyl)ethoxy]benzyl]-2,4-thiazolidinedione monohydrochloride. Pioglitazone is a potent and highly selective agonist for PPARγ and improves sensitivity to insulin in muscle and adipose tissue and inhibits hepatic gluconeogenesis. Pioglitazone improves glycemic control while reducing circulating insulin levels.

Rosiglitazone (Avandia®, GlaxoSmithKline) is a thiazolidinedione with a chemical name (RS)-5-[4-(2-[methyl(pyridin-2-yl)amino]ethoxy)benzyl]thiazolidine-2,4-dione. Like other thiazolidinediones, the mechanism of action of rosiglitazone is by activation of the intracellular PPARs, specifically PPARγ. Rosiglitazone is a selective ligand of PPARγ, and has no PPARα-binding action.

As used herein, the terms “treat”, “treating”, or “treatment” means to alleviate, reduce or abrogate one or more symptoms or characteristics of a disease and may be curative, palliative, prophylactic or slow the progression of the disease.

The term “effective amount” means an amount that will result in reduction of, as applicable or specified, damage to digestive system, and in a desired effect or result. The term “therapeutically effective amount” means an amount of a thiazolidinedione comprising, but not limited to, one or more of pioglitazone, rosiglitazone, lobeglitazone, ciglitazone, darglitazone, deuterium-stabilized R-pioglitazone, englitazone, leriglitazone, netoglitazone, rivoglitazone, troglitazone, balaglitazone, and other thiazolidinedione molecules, alone or combined with other active ingredients, that will elicit a desired biological or pharmacological response, e.g., effective to prevent, alleviate, or ameliorate symptoms of a disease or disorder; slow, halt or reverse an underlying disease process or progression; partially or fully restore cellular function; or prolong the survival of the subject being treated. In some embodiments, the thiazolidinedione is not pioglitazone.

The term “patient” or “subject” includes mammals, including non-human mammals and humans. In one embodiment the patient or subject is a human.

The term “significant” or “significantly” is determined by t-test at 0.05 level of significance.

The present methods are predicted on the surprising finding that thiazolidinediones can significantly protect digestive system against damage, as evidenced by inflammation. Therefore, disclosed herein is the use of one or more thiazolidinedione for the treatment of GI diseases.

As used herein the terms “digestive” and “gastrointestinal” are used interchangeably and refer to organs, cell, tissue, and diseases associated with the gastrointestinal (GI) tract, including but not limited to the mouth, pharynx (throat), esophagus, stomach, small intestine, large intestine, rectum, and anus, along with the salivary glands, liver, pancreas, and gallbladder.

Digestive (gastrointestinal) diseases that are driven by injury to the digestive system include, but are not limited to, abdominal adhesions, acid reflux (gastroesophageal reflux disease or GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, inflammatory bowel disease (IBD), irritable bowel syndrome (IBS), lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, Zollinger-Ellison syndrome, or a condition associated therewith.

Intestinal macrophages are key immune cells in the maintenance of intestinal immune homeostasis and have a role in the pathogenesis of GI diseases such as inflammatory bowel diseases. According to the microenvironmental cues, macrophages polarize into two different phenotypes: the classically activated (M1) or alternatively activated (M2) macrophages. For example, when the intestinal barrier function is impaired, the efferocytosis effect leads to macrophages skewing toward M2 subtypes functionally, producing cytokines, chemokines and lipid mediators that participate in healing the intestinal mucosal barrier and maintaining homeostasis. Various cytokines and other soluble factors, including prostaglandin E2 (PGE2), bone morphogenetic protein 2 (BMP2), and WNT ligands, can be produced by gut macrophages that stimulate the expansion of epithelial cells and participate in the enteric nervous system or intestinal mucosal barrier to help maintain tissue homeostasis. Intestinal macrophages maintain tolerance via promoting the proliferation of antigen-specific CD4⁺CD25⁺ regulatory T (T-reg) cells primarily by producing IL-10 in order to inhibit unrestrained inflammation as a response to innocuous commensal microbes.

The infiltration and activation of macrophages can phagocytize pathogens, as well as produce various cytokines under certain circumstances, and cooperate with distinct immune cells in many aspects of the pathogenesis of GI diseases. Disequilibrium of macrophage polarization results in the exacerbation of GI disease, and the production of particular cytokines and/or chemokines relies on the ratio of the pro-inflammatory M1 and anti-inflammatory M2 subsets. Therefore, targeted therapy of macrophages is a novel option to modulate the immune microenvironment and remodel intestinal tissue.

Gastroparesis is a disorder characterized by delayed gastric emptying (DGE) in the absence of mechanical obstruction. Symptoms are chronic with episodic exacerbation. The idiopathic form of the disorder, which accounts for the greatest number of cases, predominantly affects young adult females. Gastroparesis is also frequently associated with diabetes (diabetic gastroparesis), which likely occurs because of impaired neural control of gastric motility. Macrophages play a role in the development and progression of gastroparesis. In particular, macrophages infiltrate the smooth muscle layer of the stomach in people with gastroparesis and release pro-inflammatory molecules that contribute to the damage and dysfunction of the stomach muscles. There are two different subtypes of macrophages that have different functions in the immune system, i.e., M1 and M2 macrophages. M1 macrophages are involved in the inflammatory response and are activated in response to pro-inflammatory signals. M2 macrophages produce anti-inflammatory cytokines and growth factors that help to repair damaged tissues and promote angiogenesis. Overall, M1 and M2 macrophages represent two different functional states of macrophages that allow them to respond to different signals and perform different roles in the immune system. The balance between M1 and M2 macrophages is important in maintaining immune homeostasis and promoting proper immune responses. Drugs that can inhibit M1 macrophage, promote M2 macrophage and/or enable M1-to-M2 macrophage repolarization may be able to treat digestive diseases including gastroparesis.

Thus, the present disclosure also provides for the use of a thiazolidinedione in the inhibition or promotion of M1 macrophage differentiation, and/or promotion of M2 macrophage differentiation. In some embodiments, the thiazolidinedione is lobeglitazone.

The signs and symptoms of gastroparesis are nausea, vomiting, postprandial fullness, early satiety, and upper abdominal pain. Patients may experience any combination of signs and symptoms with varying degrees of severity. Pain is more prevalent in patients with idiopathic gastroparesis than it is in patients with diabetic gastroparesis. Patients with diabetic gastroparesis may experience further derangement of glucose control because of unpredictable gastric emptying and altered absorption of orally administered hypoglycemic drugs.

Understanding the relevant symptoms of gastroparesis is important in treating patients with this disorder. In gastroparesis, the symptom experience and severity are obtained from the patient. Consequently, patient-reported symptom scales that capture overall gastroparesis severity are necessary for evaluating treatments for gastroparesis. A well-defined patient reported outcome (PRO) instrument that measures clinically important signs and symptoms of gastroparesis would be a useful assessment tool for clinical trials to support labeling claims for treatment of gastroparesis. The American Neurogastroenterology and Motility Society Gastroparesis Cardinal Symptom Index-Daily Diary (ANMS GCSI-DD) is a patient-reported outcome instrument that captures the daily relevant symptoms of gastroparesis. The ANMS GCSI-DD is designed to assess GI symptoms associated with idiopathic and diabetic gastroparesis. The ANMS GCSI-DD is designed to assess symptoms associated with idiopathic and diabetic gastroparesis. The ANMS GCSI-DD includes 5 items, i.e., nausea, vomiting, early satiety, postprandial fullness, and upper abdominal pain. Four of these (nausea, early satiety, postprandial fullness, and upper abdominal pain) are rated as none (0), mild (1), moderate (2), severe (3), very severe (4) scale on the worst severity of the symptom over the last 24 hours. Vomiting is assessed as the number of emesis episodes over the last 24 hours, with the maximum number capped at four (0 to 4 range). The ANMS GCSI-DD total score is a useful patient reported outcome for gastroparesis and as an endpoint in gastroparesis clinical trials.

In some aspects, the present disclosure provides a method of exerting protective effects in a cell, comprising contacting the cell with an effective amount of a thiazolidinedione. As used herein, the term “effective amount” refers to an amount of thiazolidinedione that will result in the desired effect or result, e.g., an amount that will result in protective effect.

In some aspects, the disclosure provides a method of decreasing inflammation, comprising the step of contacting the cell with an effective amount of a thiazolidinedione.

In some aspects, the disclosure provides a method of increasing cell lifespan, comprising the step of contacting the cell with an effective amount of a thiazolidinedione.

In some embodiments, the cell is an animal cell, e.g., a mammalian cell. In some embodiments, the cell in a human cell or non-human cell. In some embodiments, the cell is treated in vitro, in vivo, or ex vivo. In some embodiments, the cell is a diseased cell. In some embodiments, the cell is diseased cell from a patient suffering from a disease or disorder disclosed herein.

Also disclosed herein are methods of treating a mammal having a disease or disorder that would benefit from the protective effect on cells, or for preventing or reducing the risk of acquiring a disease or disorder in an mammal, the method comprising the step of administering a therapeutically effective amount of a pharmaceutical composition comprising a thiazolidinedione to the mammal. In some embodiments, the mammal is a human or a non-human mammal. In some embodiments, the mammal is a human. In some embodiments, the disease or disorder is caused by damage that reduces function of the digestive system. In some embodiments, the disease is selected from one or more of digestive diseases, or a condition associated therewith. In some embodiments, the disease is gastroparesis. In some embodiments, the disease is idiopathic gastroparesis, diabetic gastroparesis, or mediation-induced gastroparesis.

Also disclosed herein are methods for treating a disease or disorder resulting in damage or inflammation of digestive system in a mammal, or increasing lifespan of a cell in a patient with a disease or disorder resulting in damage to digestive system.

In some embodiments, the method comprising the step of administering a therapeutically effective amount of a pharmaceutical composition comprising a thiazolidinedione to the mammal. In some embodiments, the mammal is a human or a non-human mammal. In one embodiment, the disease or disorder is selected from, but not limited to, abdominal adhesions, acid reflux (gastroesophageal reflux disease or GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, inflammatory bowel disease (IBD), irritable bowel syndrome (IBS), lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, Zollinger-Ellison syndrome, or a condition associated therewith.

Also disclosed herein are methods of treating gastroparesis. In some embodiments, gastroparesis includes idiopathic gastroparesis, diabetic gastroparesis, post-surgical gastroparesis, and medication-induced gastroparesis. In some embodiments, the primary gastroparesis is idiopathic. In some embodiments, the secondary gastroparesis is caused by a disease such as diabetes, cancer, or infections, or a drug side effect, or a surgery.

In another aspect, provided herein are methods of reducing inflammation in the GI tract for treatment of digestive diseases such as gastroparesis and IBD.

In another aspect, provided herein are methods of reducing the risk for nausea, early satiety, postprandial fullness, upper abdominal pain, vomiting episodes, and overall severity of gastroparesis.

In another aspect, provided herein are methods of slowing, stopping, or reversing disease progression to digestive disease such as gastroparesis and IBD.

In another aspect, provided herein are methods of slowing, stopping, or reversing disease progression, as indicated by nausea, early satiety, postprandial fullness, upper abdominal pain, vomiting episodes, and overall severity of gastroparesis.

In another aspect, disclosed herein are methods of treating a mammal having a disease or disorder with a symptom that is prevented, alleviated, or ameliorated by cell protection; or with a disease process or progression that slowed, halted or reversed by cell protection; the method comprising administering a therapeutically effective amount of a pharmaceutical composition comprising a thiazolidinedione. In some embodiments, the thiazolidinedione is lobeglitazone.

In a related aspect, disclosed herein are methods of treating gastroparesis. In one embodiment, gastroparesis includes idiopathic gastroparesis, diabetic gastroparesis, post-surgical gastroparesis, and medication-induced gastroparesis.

The present disclosure further provides of the use of a thiazolidinedione for the preparation of a medicament for treating a human having any one of the diseases or disorders disclosed herein or for use in any method of the present disclosure involving the administration of a thiazolidinedione to a human.

The pharmaceutical compositions of the present disclosure comprise a therapeutically effective amount of a thiazolidinedione and at least one pharmaceutically acceptable excipient. The term “excipient” refers to a pharmaceutically acceptable, inactive substance used as a carrier for the pharmaceutically active ingredient thiazolidinedione, and includes antiadherents, binders, coatings, disintegrants, fillers, diluents, solvents, flavors, bulkants, colors, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

A pharmaceutical formulation of the present disclosure may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of thiazolidinedione and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of thiazolidinedione administered to a patient in a single dose.

In some embodiments, a pharmaceutical composition disclosed herein is delivered to a subject via a parenteral route, an enteral route, or a topical route.

Examples of parental routes suitable for use with the disclosed pharmaceutical compositions include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous. In some embodiments, the route of administration is not buccal or sublingual administration. In some embodiments, the route of administration is not nasal administration.

Enteral routes of administration include administration to the GI tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories.

Topical administration includes administration to a body surface, such as skin or mucous membranes, including pulmonary administration. Transdermal forms include cream, foam, gel, lotion or ointment. Pulmonary forms include liquids and powders, e.g., liquid spray.

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of thiazolidinedione administered to a patient is selected from up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, 2 mg, 1 mg, 0.9 mg, 0.8 mg, 0.7 mg, 0.6 mg, 0.5 mg, 0.45 mg, 0.4 mg, 0.3 mg, 0.2 mg, 0.1 mg, 0.08 mg, 0.05 mg, 0.03 mg, 0.02 mg or up to 0.01 mg. In another embodiment, the daily dose is at least 0.01 mg, 0.02 mg, 0.05 mg, 0.08 mg, 0.1 mg, 0.2 mg, 0.3 mg, 0.4 mg, 0.45 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1,000 mg, 2,000 mg, 3,000 mg, 4,000 mg, or at least 5,000 mg. In another embodiment, the daily dose is 0.01-0.0.2 mg, 0.02-0.05 mg, 0.05-0.08 mg, 0.08-0.1 mg, 0.1-0.2 mg, 0.2-0.4 mg, 0.4-0.6 mg, 0.6-0.8 mg, 0.8-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, 5-200 mg, 5-300 mg, 5-400 mg, 5-500 mg, 5-600 mg, 5-700 mg, 5-800 mg, 5-900 mg, 5-1,000 mg, 5-2,000 mg, 5-5,000 mg or more than 5,000 mg, or any range bound by a pair of these values.

In another embodiment, a single dose of thiazolidinedione administered to a patient is selected from: 0.01 mg, 0.02 mg, 0.05 mg, 0.08 mg, 0.1 mg, 0.2 mg, 0.3 mg, 0.4 mg, 0.45 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg 490 mg, 500 mg, 600 mg, 700 mg, 800 mg, 900 mg, 1,000 mg, 2,000 mg, 3,000 mg, 4,000 mg, or 5,000 mg, or any range bound by a pair of these values. In one embodiment, the single dose is administered by oral administration. In another embodiment, the single dose is administered by injection, e.g., subcutaneous, intramuscular, or intravenous. In another embodiment, the single dose is administered by inhalation administration. In some embodiments, the thiazolidinedione is lobeglitazone.

As a non-limited example, the dose of a thiazolidinedione, such as lobeglitazone, administered by oral administration may be about 0.01 to 50 mg per day to be administered in divided doses. A single dose of a thiazolidinedione, such as lobeglitazone, administered by subcutaneous injection may be about 0.01-50 mg, preferably about 0.1-10 mg, 0.2-1 mg, 0.3-0.6 mg or 0.5 mg, or any range bound by a pair of these values. Other embodiments include ranges of about 0.05-5,000 mg, preferably about 0.1-10 mg, 0.2-5 mg, 0.3-1 mg, or 0.5 mg, or any range bound by a pair of these values. Subcutaneous infusion may be preferable in those patients requiring division of injections into more than 10 doses daily.

The fine particle dose of a thiazolidinedione, such as lobeglitazone, administered by pulmonary administration, e.g., inhalation using a pressurized metered dose inhaler (pMDI), dry powder inhaler (DPI), soft-mist inhaler, nebulizer, or other device, may be in the range of about, 0.1-50 mg, preferably about 0.2-10 mg, 0.3-1 mg, or 0.5 mg, or any range bound by a pair of these values. Other embodiments include ranges of about 0.05-5,000 mg, preferably about 0.1-1,000 mg, 0.2-100 mg, 0.3-1 mg, 0.4-0.5 mg, or 0.5 mg, or any range bound by a pair of these values. The Nominal Dose (ND), i.e., the amount of drug metered in the receptacle (also known as the metered dose), of a thiazolidinedione, such as lobeglitazone, administered by pulmonary administration may be, for example, in the range of 0.1-15 mg, 0.1-10 mg, 0.1-1 mg, 0.2-0.3 mg, 0.3-0.4 mg, 0.4-0.5 mg, 0.5-0.6 mg, 0.6-0.7 mg, 0.7-0.8 mg, 0.8-0.9 mg, or 0.9-1 mg, or any range bound by a pair of these values. Other embodiments include ranges of about 0.05-5,000 mg, preferably about 0.1-1,000 mg, 0.2-10 mg, 0.3-1 mg, 0.4-0.5 mg, or 0.5 mg, or any range bound by a pair of these values.

Long-acting pharmaceutical compositions may be administered, 1, 2, 3, 4, 5, 6, 7, 8, 9, 10 or more than 10 times daily (preferably ≤10 times per day), every other day, every 3 to 4 days, every week, or once every two weeks depending on half-life and clearance rate of the particular formulation.

In an embodiment of any of the above methods and compositions, a thiazolidinedione, or its salt, solvates, hydrates, and co-crystals thereof, is a racemic mixture of R and S enantiomers, or enriched in R enantiomer (i.e., the ratio of R to S enantiomer being administered, is from 1.1:1 to 1,000:1, from 10:1 to 10,000:1, or from 100:1 to 100,000:1, or over all thiazolidinedione enantiomers in the composition is at least 98% R enantiomer, 99% enantiomer, 99.5% enantiomer, 99.9% enantiomer, or is free of any observable amount of S enantiomer), or enriched in S enantiomer (i.e., the ratio of S to R enantiomer is from 1.1:1 to 1,000:1, from 10:1 to 10,000:1, or from 100:1 to 100,000:1, or over all thiazolidinedione enantiomers in the composition is at least 98% S enantiomer, 99% enantiomer, 99.5% enantiomer, 99.9% enantiomer, or is free of any detectable amount of R enantiomer).

The present disclosure further provides an in vitro or ex vivo method of reducing cell damage, the method comprising the step of contacting the cell with an effective amount of a thiazolidinedione.

Suitably, the cell is having a disease or disorder or at risk of the disease or disorder or at risk of acquiring the disease or disorder selected from any one or more of: abdominal adhesions, acid reflux (gastroesophageal reflux disease or GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, inflammatory bowel disease (IBD), irritable bowel syndrome (IBS), lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, Zollinger-Ellison syndrome, or a condition associated therewith.

Suitably, in the methods, composition and/or second medical uses of the presently disclosed compositions, a thiazolidinedione may be administered or formulated for administration at a dose of 0.01 mg or higher. Suitably, a thiazolidinedione is administered at a dose between 0.1-5000 mg/day.

Suitably, in the methods, composition and/or second medical uses of the presently disclosed compositions, a thiazolidinedione may be administered or formulated for administration in any suitable way, for example parenterally, enterally, or topically.

Suitably, in the methods, composition and/or second medical uses of the presently disclosed compositions, a thiazolidinedione may be administered or formulated for administration by oral, sublingual, buccal, pulmonary, intravenous, intramuscular, or subcutaneous administration.

Another embodiment of the present disclosure includes use of a thiazolidinedione to decrease the cell damage or improve cell survival.

Another embodiment of the present disclosure includes use of a thiazolidinedione to treat gastroparesis or IBD.

Another embodiment of the present disclosure includes use of a thiazolidinedione to decrease core signs and symptoms of gastroparesis, measured by ANMS GCSI-DD.

Another embodiment of the present disclosure includes use of a thiazolidinedione to decrease the severity of nausea.

Another embodiment of the present disclosure includes use of a thiazolidinedione to decrease the severity of early satiety.

Another embodiment of the present disclosure includes use of a thiazolidinedione to decrease the severity of postprandial fullness.

Another embodiment of the present disclosure includes use of a thiazolidinedione to decrease the severity of upper abdominal pain.

Another embodiment of the present disclosure includes use of a thiazolidinedione to decrease the severity of vomiting episodes.

Another embodiment of the present disclosure includes use of a thiazolidinedione to decrease the overall severity of gastroparesis.

### EXAMPLES

The present invention is not to be limited in scope by the specific embodiments described herein. Indeed, various modifications of the invention in addition to those described herein will be apparent to those skilled in the art from the foregoing description and the accompanying Figures. Such modifications are intended to fall within the scope of the appended claims.

It is further to be understood that all values are approximate and are provided for description. All references cited and discussed in this specification are incorporated herein by reference in their entirety and to the same extent as if each reference was individually incorporated by reference.

### Example 1: Efficacy of Lobeglitazone to Treat GI Inflammation

### Methodology

The purpose of this study was to test the efficacy of compound lobeglitazone in the DSS-induced IBD model in female Balb/c mice following oral administration. The study was conducted at an AAALAC accredited facility, and all animal study procedures were approved by the Institutional Animal Care and Use Committee (IACUC).

This study was conducted according to the procedures outlined below.

### Preparation of Vehicle and Test Articles

Totally 5 g carboxymethyl cellulose (CMC) powder was weighed out and transferred into a clean bottle, 1 L Milli Q water was added subsequently. Then stir bar was added, the solution was stirred over 6 hours until it was clear. The 0.5% CMC was stored at 4° C. for further usage.

Precise amount of lobeglitazone sulfate (lot: EW34258-13-P1; purity: 97.6%) was weighted out into a clean tube, 0.5% CMC was added and vortexed fully, then sonicated 2-3 minutes to get an even suspension. The concentration of the solution was 0.361 mg/ml, 0.120 mg/mL, and 0.036 mg/mL. The dosing solution was freshly made daily until the end of the study. The compound was orally (PO), 10 mL/kg daily dosed for 10 days.

Totally 125.82 mg cyclosporin A (CSA, purity 98%) was weighed out into a clean tube, 2.055 mL castor oil was added firstly, then 39.046 mL 95% CMC (with 0.1% tween80) was added, vortexed fully, then sonicated 10 min to get a clear solution. The concentration of the solution was 3 mg/mL. The compound was PO, 10 mL/kg daily dosed for 10 days.

5% DSS preparation: 5 g DSS powder was weighted out into a clean bottle, 1 L Milli Q water was added subsequently, the solution was stirred until it was clear. Then 5% DSS was transferred to water bottle of the animal. 5% DSS was freshly made daily until the end of the study.

### Animal Husbandry

A total of 50 (7 weeks old) female Balb/c were purchased from Beijing Vital River Laboratory Animal Technology Co., Ltd. Animals were acclimatized for 1 week prior to the experiment. All the in vivo experimental procedures were approved by the institutional animal care and use committee (IACUC). All euthanasia was performed using carbon dioxide inhalation and all efforts were made to minimize animal suffering.

### Group and Dose Design

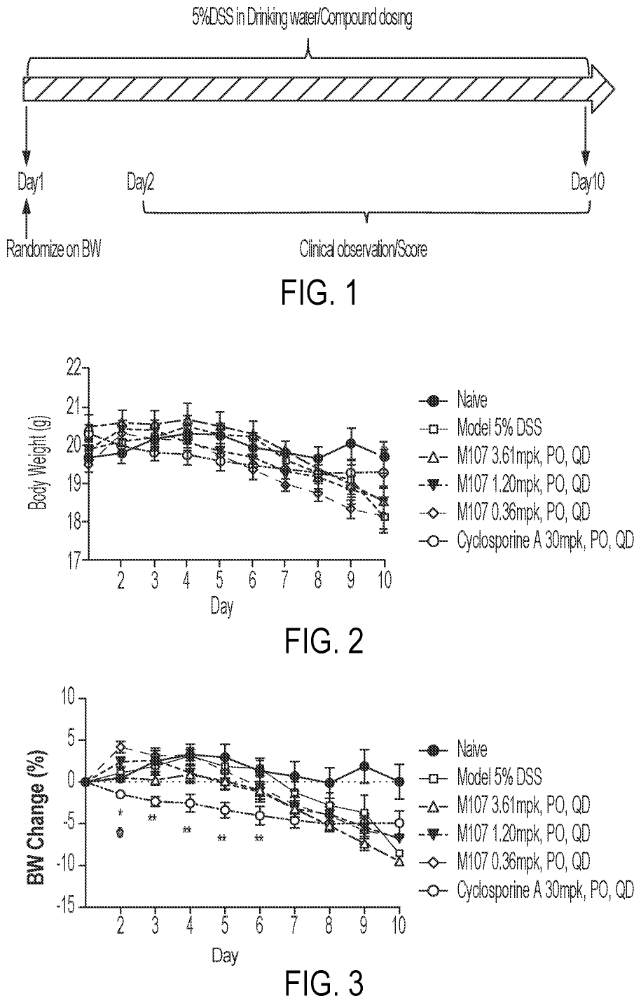

At the beginning of the study, randomization was performed based on body weight. All animals were randomly assigned to one of six groups (Table 1). The study schedule is described in FIG. 1.

TABLE 1
Study groups and dose design

| | | | | | Dosing | |
|---|---|---|---|---|---|---|
| | | | | | Route & | Dosing |
| | | | Disease | Compound | Fre- | Level & |
| Group | N | Gender | Induction | Treatment | quency | Volume |
| 1 | 7 | F | N/A | N/A | N/A | N/A |
| 2 | 8 | F | 5% DSS | Vehicle | PO; QD | N/A |
| | | | for 10 | | | |
| | | | days | | | |
| 3 | 9 | F | 5% DSS | lobeglitazone | PO; QD | 3.61 mg/ |
| | | | for 10 | | | kg/day; |
| | | | days | | | 10 mL/kg |
| 4 | 9 | F | 5% DSS | lobeglitazone | PO; QD | 1.20 mg/ |
| | | | for 10 | | | kg/day; |
| | | | days | | | 10 mL/kg |
| 5 | 9 | F | 5% DSS | lobeglitazone | PO; QD | 0.36 mg/ |
| | | | for 10 | | | kg/day; |
| | | | days | | | 10 mL/kg |
| 6 | 8 | F | 5% DSS | Cyclosporin | PO; QD | 30 mg/kg; |
| | | | for 10 | A | | 10 mL/kg |
| | | | days | | | |

### Monitoring of Animal Health

During the study, the general condition (appearance and activity) of all animals were carefully monitored daily by the veterinarian.

### Disease Activity Index (DAI) Score

The DAI was evaluated using the criteria listed in Table 2. DAI score was calculated by the sum of weight loss score, stool consistency score, and occult-gross bleeding score. Body weight loss was calculated by comparison with the body weight on day 1. Feces samples were collected from each individual animal, and stool consistency and occult-gross bleeding were evaluated according to Table 2. Stool Consistency was evaluated by the appearance of the feces. Occult-gross bleeding was evaluated using Fecal Occult Blood test strips. Occult bleeding score of 1 will be assigned if light or dark purple color developed within 20 seconds and longer than 15 seconds. Occult bleeding score of 2 was assigned if light or dark purple color developed within 15 seconds. Occult-gross bleeding score of 3 would be assigned if blood was visible in the feces.

TABLE 2
DAI Score.

| | Weight Loss | Stool | |
|---|---|---|---|
| Score | (%) | Consistency | Occult-gross Bleeding |
| 0 | None | Normal | None |
| 1 | 1-5 | | Mild Occult Blood (+) |
| | | | (Occult blood test positive, |
| | | | developing ≤20 seconds |
| | | | and >15 seconds) |
| 2 | 5-10 | Loose Stool | Occult Blood (+) |
| | | | (Occult blood test positive, |
| | | | developing ≤ 15 seconds) |
| 3 | 10-15 | | |
| 4 | >15 | Diarrhea | Gross Bleeding |

### Sample Collection

Animals were euthanized by CO₂ at the end of study. Colon length and colon weight (cecum is not included) were recorded, and macroscopic changes were scored based on Table 2. The colon was cleaned using saline, and all feces were removed before weight measurement. The middle part of colon was collected in cassette and fixed in 10% neutral buffered formalin (NBF) for H&E and PAS staining. For left colon, mucosal from colon surface was removed and then snap frozen in liquid nitrogen and store at −80° C. for QT-PCR analysis of TNF-α, IL-10, CCL2, and IL-1β. QT-PCR primers were purchased from Sangon Biotech Co., Ltd.

Treatment of Tissue before RNA Extraction are as follows.
(a) Remove mucosal from colon surface. Cut colon tissue into small pieces on ice.
(b) Weight out the tissue no more than 30 mg and transfer the tissue into a lysing matrix D tube. Totally 700 UL Trizol was added in the tissue and lysing matrix D immediately.
(c) Put the lysing matrix D tubes which contain tissue, Lysing matrix, and 700 μL Trizol into the FastPrep™-24 Sample Preparation System.
(d) Set at speed 6 and grind for 2 min
(e) 140 μL chloroform was added into the tubes, shake the tubes vigorously by vortex for 15 seconds. Incubate at room temperature for 2 min.
(f) 12000 rpm centrifuge for 10 min at 4° C.,
(g) Pipet the supernatant to 96 well 2.2 mL deep well plate

RNA Sample Preparation procedure is as follows.
(a) 600 μL Binding Buffer was added into column 1 and 7
(b) 600 μL DNase Stop Buffer was added into column 2 I 8
(c) 600 μL Wash Buffer was added into column 3, 4 and 9, 10
(d) 100 μL Elution Buffer was added into column 5Id 11
(e) 190 μL DEPC water and 10 μL MagaBio® Reagent was added into column 6 and 12
(f) Transfer 300 μL of the colorless, upper phase containing the RNA to column 1 and 7
(g) Put the test plate in nucleic acid purifier
(h) Set the instrument as below in Table 3.

TABLE 3

| | | | Waiting | Mixing | Magnet | | | |
|---|---|---|---|---|---|---|---|---|
| | | | Time | Time | Time | Ad- | | Vol- |
| | | | (min: | (min: | (min: | sorp- | | ume |
| Step | Well | Name | sec) | sec) | sec) | tion | Speed | (μL) |
| 1 | 1 | Mixing | 0:00 | 0:30 | 0:00 | | F | 900 |
| 2 | 6 | Beads | 0:00 | 0:15 | 0:30 | | M | 200 |
| 3 | 1 | Binding | 0:00 | 10:00 | 0:35 | √ | F | 900 |
| 4 | 2 | Wash 1 | 0:00 | 2:00 | 0:30 | √ | F | 600 |
| 5 | 3 | Wash 2 | 0:00 | 1:00 | 0:30 | √ | F | 600 |
| 6 | 4 | Wash 3 | 0:00 | 1:00 | 0:30 | √ | F | 600 |
| 7 | 5 | Elution | 1:00 | 5:00 | 0:30 | | M | 50 |
| 8 | 6 | Beads | 0:00 | 0:30 | 0:00 | | M | 200 |

The elution was transferred into a clean nuclease-free centrifuge tube and the RNA sample was store at −80° C.

To use the Takara Prime Script™ Reverse Transcription System, Mix and briefly centrifuge each component before use. Combine the following component volume as in Table 4.

TABLE 4

| Reaction Component | Volume (μL) |
|---|---|
| 5 x gDNA Eraser Buffer | 2 |
| gDNA Eraser | 1 |
| Total RNA 1 μg | x |
| Nuclease-Free Water | x |
| Final volume | 10 |

(a) Store on ice until reverse transcription mix is added. Centrifuge 10 seconds in a micro-centrifuge. Heat in a 42° C. heat block for 2 minutes, 4° C. immediately chill in ice water for at least 5 minutes.
(b) Prepare the reverse transcription reaction mix, 10 μL for each cDNA reaction. Combine on ice, in the order listed. Component volume of step b) (Table 5).

TABLE 5

| Reaction Component | Volume (μL) |
|---|---|
| 5x PrimeScript buffer | 4 |
| PrimeScript RT Enzyme Mix | 1 |
| RT primer Mix | 1 |
| Nuclease-Free Water | 4 |
| Final volume | 10 |

(c) Combine 20 μL of reverse transcription mix, Centrifuge 10 seconds in a microcentrifuge.
(d) Anneal in a heat block at 37° C. for 15 min, 85° C. for 5 sec, 4° C. save on S100™ Thermal Cycler.
(e) Aliquot the cDNA sample and store at −20° C.

### QT-PCR Procedure

(a) Make qPCR reaction mixture in one tube based on assay number and replicas.
(b) Prepare primers and master mix mixture according to manufacturer's instructions. And aliquot 9 μL of mixture into each well of the 384 qPCR plate.
(c) Transfer 1 μL of cDNA into each well of a 384 qPCR plate which already contain 9 μL of mixture. Briefly centrifuge the plate.
(d) Seal the plate
(e) Mix thoroughly, spin briefly
(f) Run the PCR Reaction
(g) Place the reaction plate in the instrument.
(h) Set the Applied Biosystems ViiA™ 7 cycling conditions as in Table 6.

TABLE 6
Times and Temperatures

| Initial | PCR (40 Cycles) | | | | |
|---|---|---|---|---|---|
| Steps | | Anneal/ | | | |
| Denature | Melt | Extend | Dissociation Steps | | |
| HOLD | CYCLE | | HOLD | HOLD | HOLD |
| 30 sec | 5 sec | 34 sec | 15 sec | 1 min | 15 sec |
| 95° C. | 95° C. | 60° C. | 95° C. | 60° C. | 95° C. |

### Statistics for Real-Time Quantitative QT-PCR

Data were expressed as mean±SD. The 2-ΔΔCT method was used to analyze the mRNA expression level. The results were normalized for RNA input using glyceraldehydes-3-phosphate dehydrogenase (GAPDH or other internal controls). Statistical analysis was performed using, one-way ANOVA followed if significant by post-hoc Dunnett test. Nonparametric test like Mann-Whitney and Kruskal-Wallis test were used when N was too small or data did not follow Gaussian distribution. The difference was considered significant when p<0.05

### Histology Analysis

H&E staining: Colon sections were cut at 4 μm in thickness, dried in an oven for 1 hour and stained with H&E solution with our standard protocol. Briefly, the sections were stained with hematoxylin solution for 90 seconds. Then stain in eosin solution for 30 seconds. Then dehydrated and coverslipped for the subsequent image analysis. H&E stained images was scanned using LEICA Aperio® GT450. The severity of inflammatory cell infiltration and tissue damage were semi-quantitatively evaluated according to pathology score criteria in Table 7.

TABLE 7

| Score | Inflammatory cell infiltration | Tissue Damage |
|---|---|---|
| 0 | Occasional or no infiltrate | No mucosal damage |
| 1 | Infiltration into lamina propria | Focal crypt lesions |
| 2 | Infiltration into submucosa | Mucosal erosions or |
| | | ulcerations |
| 3 | Transmural infiltration | Extensive damage |
| | | affecting the submucosa |

PAS staining: Colon sections were cut at 4 μm in thickness, dried in an oven for 1 hour and stained with PAS kit (Gefan #M035) with our standard protocol. Briefly, the sections were stained with periodic acid working solution for 8 minutes. Then wash with distilled water for 5 minutes, three times. Stain in Schiff solution for 10 minutes. Transform to distilled water for 5 minutes, three times. Then dehydrated and coverslipped for the subsequent image analysis.

The total length of the colon was measured by a ruler and then was divided into 3 segments, 100 microns in the middle of each segment was taken out for goblet cell calculation. In each 100 microns of colon tissue, the number of goblet cells was calculated, then the average number of goblet cells from three parts was used for next analysis.

### Data Analysis

In vivo animal data were presented as mean±SEM. Statistical analysis was performed by one-way ANOVA followed by Dunnett's multiple comparison. Nonparametric tests Whitney etc., were used when the N was too small, or data did not follow Gaussian distribution. The difference was considered significance when p<0.05.

### Results

### Body Weight and Body Weight Changes

As shown in FIG. 2, body weight showed no significant difference among all groups, while as shown in FIG. 3, body weight changes significantly reduced post 3.61 mg/kg lobeglitazone treatment on day 2 and 30 mg/kg CSA treatment on day 2, day 3, day 4, day 5 and day 6 vs control (no drug) group mice. There were no significant body weight changes after 1.20 mg/kg and 0.36 mg/kg lobeglitazone treatment vs control group mice.

### DAI Score

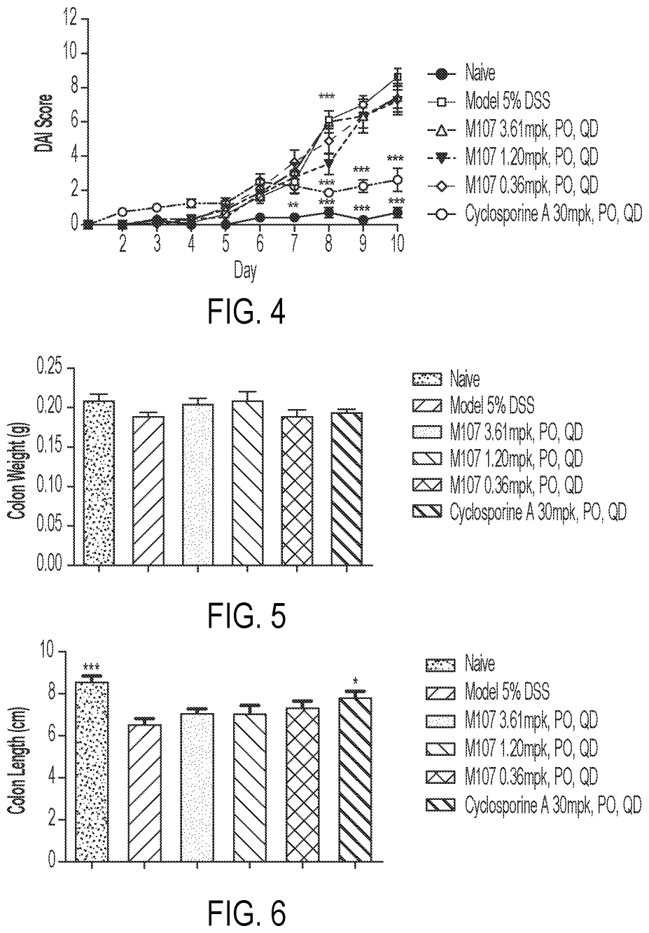

As shown in FIG. 4, DSS treatment significantly increased the mice DAI on day 7, day 8, day 9 and day 10. Compared to the control group, treatment with 30 mg/kg CSA significantly decreased mice DAI on day 8, day 9 and day 10. Treatment with lobeglitazone did not significantly reduce the animal DAI score.

### Colon Length and Colon Weight

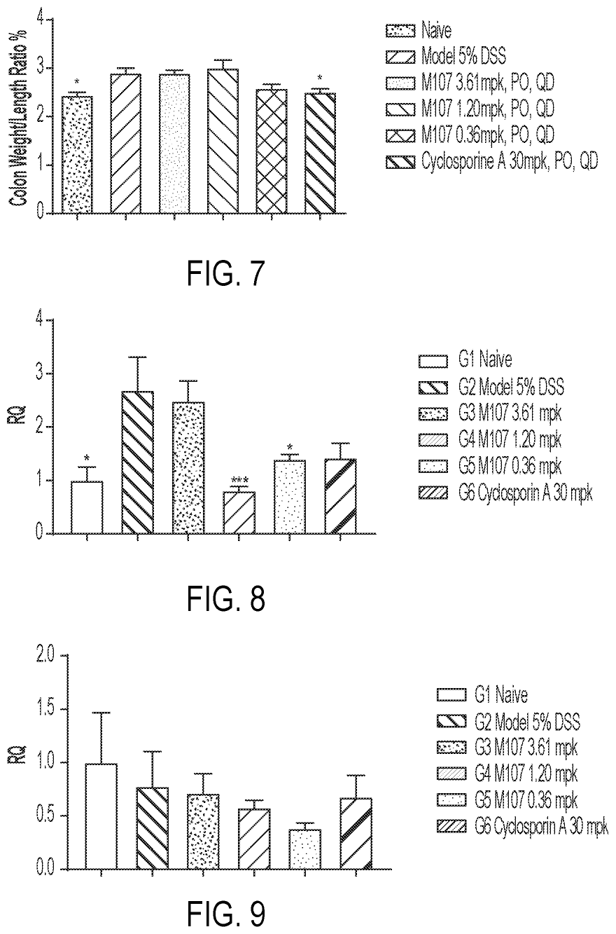

As shown in FIG. 5, DSS treatment did not significantly change colon weight. Compared to the control group, treatment with 30 mg/kg CSA and lobeglitazone did not significantly change colon weight. As shown in FIG. 6, DSS treatment significantly decreased colon length. Compared to the control group, treatment with 30 mg/kg CSA significantly increased colon length. Treatment with lobeglitazone did not significantly increase colon length. As shown in FIG. 7, DSS treatment significantly increased colon length/weight ratio. Compared to the control group, treatment with 30 mg/kg CSA significantly decreased colon length/weight ratio. Treatment with lobeglitazone did not significantly decrease colon length/weight ratio.

### Gene Express Level in Colon

As shown in FIG. 8, TNF-α gene expression levels significantly increased in colon after DSS treatment compared to naïve group. Treatment with lobeglitazone (0.36 and 1.20 mg/kg) significantly decreased TNF-α gene expression levels. Treatment with 30 mg/kg CSA decreased TNF-α expression. Treatment with lobeglitazone (3.61 mg/kg) didn't show obvious changes on TNF-α gene expression levels.

As shown in FIG. 9, IL-10 gene expression levels decreases after DSS treatment compared to naïve group. It did not change significantly after lobeglitazone and 30 mg/kg CSA treatment.

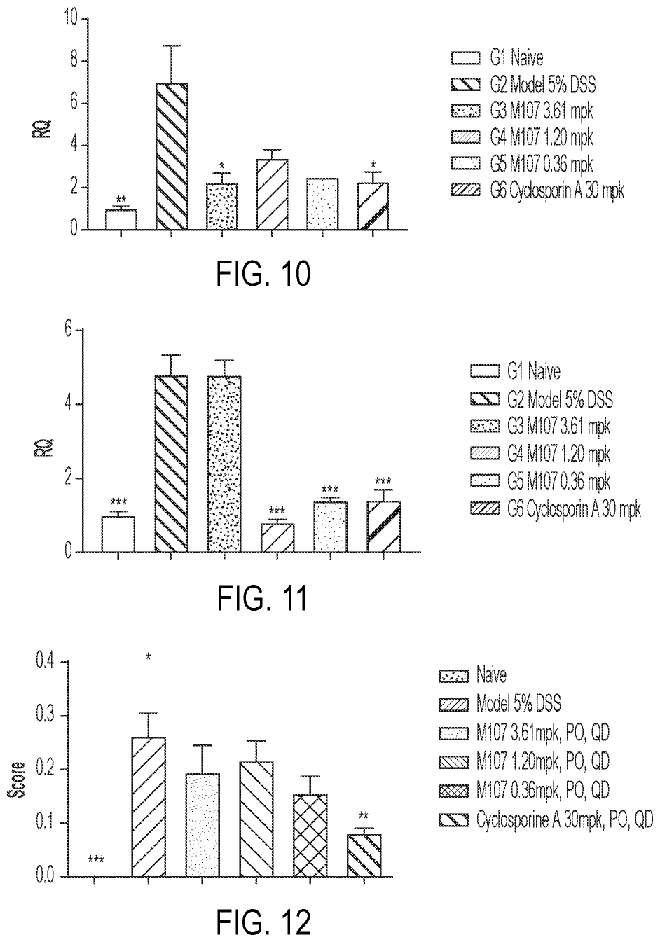

As shown in FIG. 10, CCL2 gene expression levels significantly increased in colon after DSS treatment compared to naïve group. CCL2 gene expression levels significantly decreased after 3.61 mg/kg lobeglitazone and 30 mg/kg CSA treatment. Treatment with lobeglitazone (0.36 and 1.20 mg/kg) led to decreases in levels of CCL2 gene expression.

As shown in FIG. 11, IL-1B gene expression levels significantly increased in colon after DSS treatment compared to naïve group. IL-1B gene expression levels significantly decreased after compound lobeglitazone (0.36 and 1.20 mg/kg) and 30 mg/kg CSA treatment, while 3.61 mg/kg lobeglitazone treatment did not change the levels of IL-1β gene expression.

### Colon H&E Staining and Score

The naive group showed intact mucosal structure and rich goblet cells, and the 5% DSS induced model group showed severe inflammatory cells infiltration, gland separation, crypt loss and damage of mucosal structure and significantly increased H&E total score. Treatment with 30 mg/kg CSA significantly decreased the H&E total score. Treatment with lobeglitazone (0.36, 1.20 and 3.61 mg/kg) led to decrease in the H&E total score (FIG. 12).

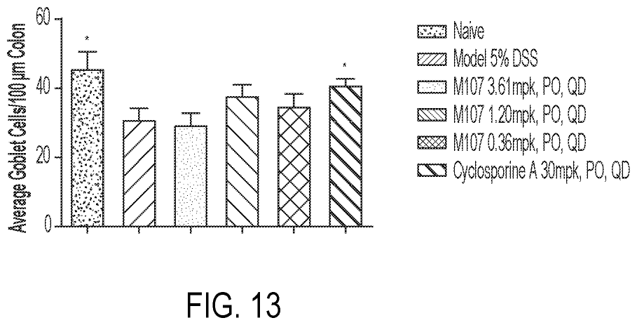

Compared to the naïve group, goblet cells were significantly reduced in the DSS-treated group. Goblet cells significantly increased after 30 mg/kg CSA treatment. Treatment with compound lobeglitazone (0.36 and 1.20 mg/kg) trended to increase the number of goblet cells, treatment with 3.61 mg/kg lobeglitazone did not change the number of goblet cells (FIG. 13).

### CONCLUSION

Lobeglitazone was evaluated in a mouse IBD model (DSS model) at 0.36 and 1.20, and 3.61 mg/kg. It was demonstrated that treatment with lobeglitazone significantly decreased GI inflammation, as evidenced by the gene expression data. Lobeglitazone did not significantly decrease the DAI score and colon weight/length ratio.

### Example 2: M1 Macrophage Differentiation Assay

### Methodology

The purpose of this study was to evaluate the effects of lobeglitazone and other thiazolidinediones on M1 macrophage differentiation. The test articles and vehicles used in this study were lobeglitazone sulfate, pioglitazone HCl, and rosiglitazone.

Media and solution reconstitution: (1) Prepare complete culture medium: add 50 mL of HI FBS (10%) and 5 mL (1%) of 100× Pen/Strep into 500 ml bottle of RPMI-1640; (2) Reconstitute LPS with sterilized water to 1 mg/ml stock concentration; (3) Reconstitute human IL-4 by solubilizing in sterilized water for 0.1 mg/ml by pipetting mix; (4) Reconstitute human M-CSF by solubilizing in sterilized water for 0.1 mg/ml by pipetting mix; (5) Reconstitute human GM-CSF by solubilizing in sterilized water for 0.1 mg/ml by pipetting mix; (6) Reconstitute test article (M107, pioglitazone, rosiglitazone) with DMSO to reach 10 mM stock concentration.

M1 macrophage differentiation assay timeline was as follows in Table 8.

TABLE 8

| Day | Action Items |
|---|---|
| Day 0 | Isolate monocytes from frozen PBMC or revive |
| | frozen monocytes |
| | Differentiate monocytes into M1/M2 macrophage |
| | Add test compound |
| Day 5 | stimulate the cells with activator for 2 days, |
| | add compound again |
| Day 7 | Collect supernatants from 96-well plates for |
| | cytokine analysis |

Drug concentrations tested in the M1 differentiation assay are as in Table 9.

TABLE 9

| Drug | Testing concentration (μM) |
|---|---|
| Lobeglitazone sulfate | 0, 0.025, 0.05, 0.1, 0.2, 0.4, |
| | 0.8, 1.6, 3.2, 6.4 |
| Pioglitazone | 0, 2.5, 5, 10, 20, 40 |
| Rosiglitazone | 0, 0.4, 0.8, 1.6, 3.2, 6.4 |

### Experimental Procedures

(1) Revive frozen monocytes.
(2) Count cells and adjust the cell density to 0.5×10⁶ cells/ml with fresh complete differentiation medium (RPMI1640+10% FBS+1% PS plus GM-CSF or M-CSF at 50 ng/ml).
(3) Seed isolated monocyte 100 μL per well on 96-well plate.
(4) Add test articles as the plate map using D300e (final concentration in μM).
(5) Differentiate the monocyte to macrophage in the presence of GM-CSF to M1, or M-CSF to M2 for 5 days.
(6) On day 5, add cytokines at 2× final concentration.
(6a) Prepare medium containing GM-CSF for M1 and M-CSF for M2.
(6b) Add corresponding activator to reach 2× final working concentration: (i) IFNγ stock 0.2 mg/ml: add 2.5 μL stock into 5 ml complete differentiation medium for 100 ng/ml; (ii) LPS stock 1.0 mg/ml: dilute to 20 μg/ml by mixing 2 μL stock into 100 μl medium; then add 10 μl 20 μg/ml LPS to 5 ml complete differentiation medium for 40 ng/ml; (iii) IL-4 stock 0.1 mg/ml: dilute 2 μL stock into 5 ml complete differentiation medium for 40 ng/ml.
(6c) Add 100 μL cytokine containing differentiating medium to each well, and total volume of each well is 200 μL.
(6d) Add test compound again using D300e. Make sure to set the final volume as 200 μL.
(7) Incubate cytokine and test compound for 48 hours.
(8) On day 7, spin down the plates at 300×g for 5 min and collect the supernatant for cytokine quantification using MSD.
(9) M1 associated macrophage cytokines TNF-α, IL-1b, IL-6, IL-12, and IL-23 were quantified.

### Results

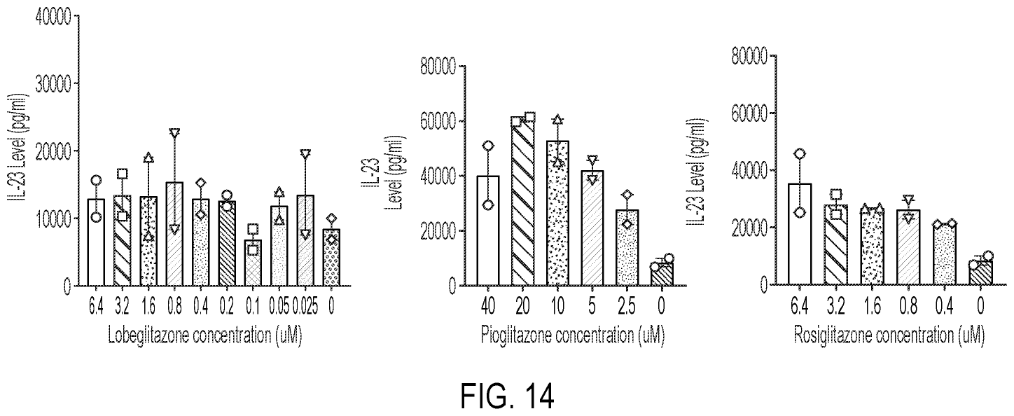

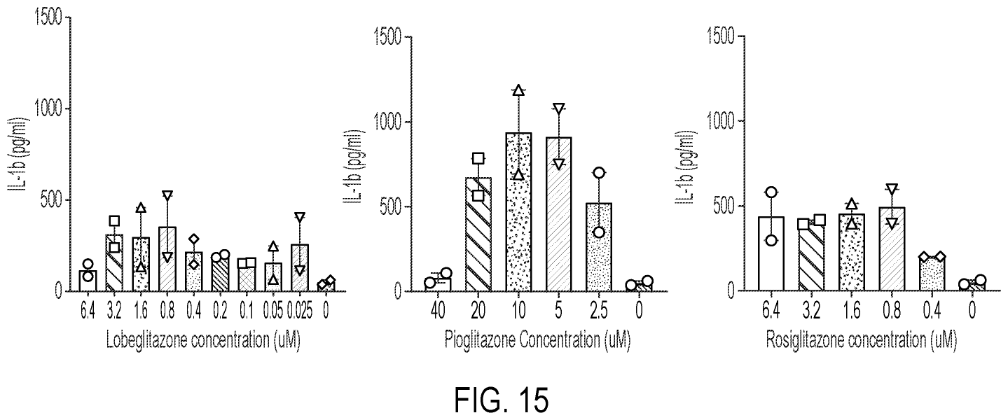

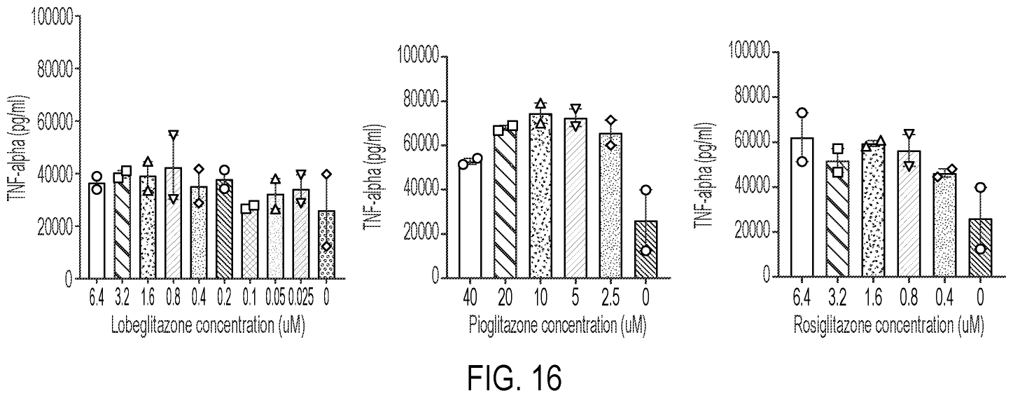

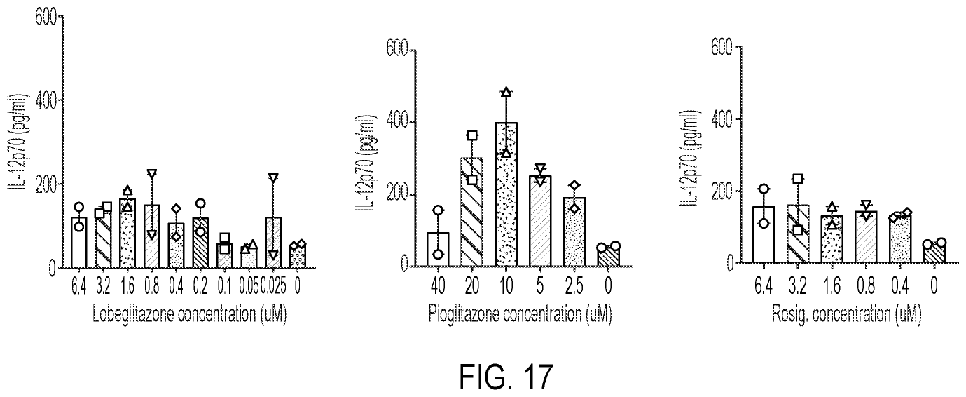

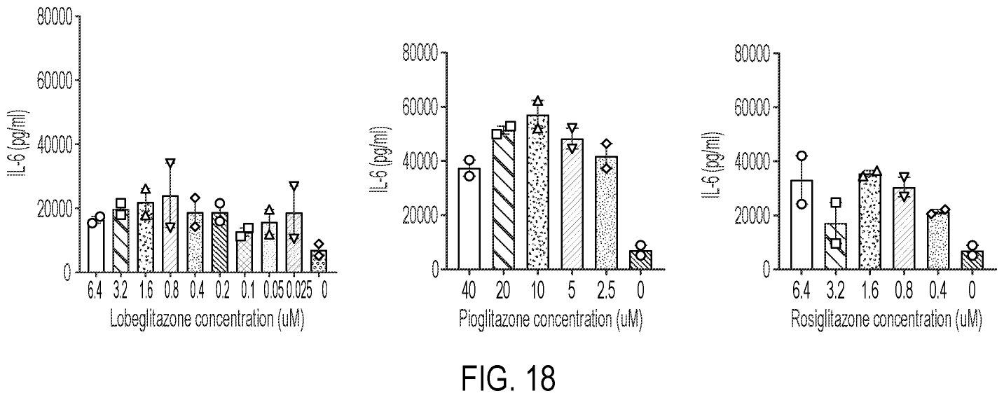

M1 macrophage cytokines expression assay results are presented in FIG. 14. M1-like macrophage cytokines expression assay results are presented in FIG. 14-18.

### CONCLUSION

This study evaluated the effects of lobeglitazone (M107), pioglitazone (Pigl), and rosiglitazone (Rosig) on M1 and M2 macrophage differentiation using cytokine assays. Among three test articles, pioglitazone and rosiglitazone promoted the M1 macrophage cytokines in a dose-dependent manner, while lobeglitazone did not show the promotion of M1 macrophage cytokines.

### Example 3: M2 Macrophage Differentiation Assay

### Methodology

The purpose of this study was to evaluate the effects of lobeglitazone and other thiazolidinediones on M2 macrophage differentiation. The test articles and vehicles used in this study were lobeglitazone sulfate, pioglitazone HCl, and rosiglitazone.

Media and solution reconstitution: (1) Prepare complete culture medium: add 50 mL of HI FBS (10%) and 5 mL (1%) of 100× Pen/Strep into 500 ml bottle of RPMI-1640; (2) Reconstitute LPS with sterilized water to 1 mg/ml stock concentration; (3) Reconstitute human IL-4 by solubilizing in sterilized water for 0.1 mg/ml by pipetting mix; (4) Reconstitute human M-CSF by solubilizing in sterilized water for 0.1 mg/ml by pipetting mix; (5) Reconstitute human GM-CSF by solubilizing in sterilized water for 0.1 mg/ml by pipetting mix; (6) Reconstitute test article (M107, pioglitazone, rosiglitazone) with DMSO to reach 10 mM stock concentration.

M2 macrophage differentiation assay timeline was as follows in Table 10.

TABLE 10

| Day | Action Items |
|---|---|
| Day 0 | Isolate monocytes from PBMC and differentiate |
| | into M2 macrophage |
| | Add test compound |
| Day 4 | Add additional medium containing M-CSF |
| | Add additional test compound (D300e final |
| | volume = 100 μl) |
| Day 6 | stimulate the cells with IL-4 for 2 days |
| Day 8 | Collect supernatants from 96-well plates for |
| | cytokine analysis |
| | Collect cells for flow (viability/CD11c/CD163/CD206) |

Drug concentrations tested in the M2 differentiation assay are shown in Table 11.

TABLE 11

| Drug | Testing concentration (μM) |
|---|---|
| Lobeglitazone sulfate | 0.025, 0.05, 0.1, 0.2, 0.4, 0.8, 1.6 |
| Pioglitazone | 2.5, 5, 7.5, 10 |
| Rosiglitazone | 0.4, 0.8, 1.6, 3.2 |

### Experimental Procedures (Monocyte Isolation, Treatment and Plating)

(1) Isolate monocytes from PBMC using StemCell® monocyte isolation kit
(2) Count cell and adjust the cell density to 0.5×10⁶ cells/ml with fresh complete differentiation medium. Add 5 μL M-CSF stock (100 μg/ml final concentration) to each 5 ml cell mixture.
(3) Seed isolated monocyte 100 μL per well on 96-well plate.
(4) Add test articles as the plate map using D300e (final concentration in μM).
(5) Differentiate the monocyte to macrophage in the presence of M-CSF to M4 for 4 days.
(6) On day 4, add additional medium containing M-CSF.
(7) Add test compounds using D300e (final volume=100 μL).
(8) On day 6, add IL-4 at 5× final concentration (IL-4 stock 0.1 mg/ml: dilute 10 μl stock into 10 ml medium for 100 ng/ml. Add 50 μl 100 ng/ml IL-4 to each well containing 200 μL medium to reach 20 ng/ml).
(9) On day 8, spin down the plates at 300×g for 5 min and collect the supernatant for cytokine quantification using MSD.
(10) M2 associated macrophage cytokines of interest are listed below in Table 12.

TABLE 12

| | Cytokines of interest |
|---|---|
| Cytokines | IL-1RA, IL-1a, IL-1b, IL-6, IL-10, IL-12p70, |
| | TNF-alpha, TNF-beta |
| Chemokines | CCL1 (1-309), CCL2 (MCP-1), CCL5 (RANTES; R-plex), |
| | CCL17 (TARC), CCL22 (MDC), CCL24 (eotaxin-2) |

(11) Collect cells for flow analysis of viability/CD11c/CD163/CD206.

### Results

Flow cytometry and surface marker CD163, CD11c, and CD206 were applied to characterize M2 repolarization. No stain and all stain with surface marker antibody to confirm the optimization of gating strategy.

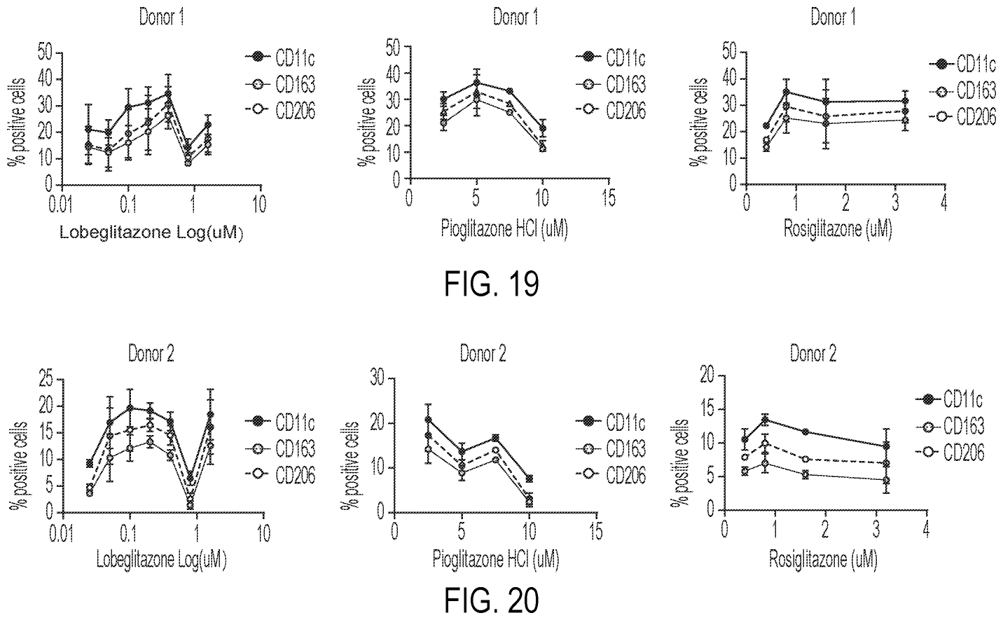

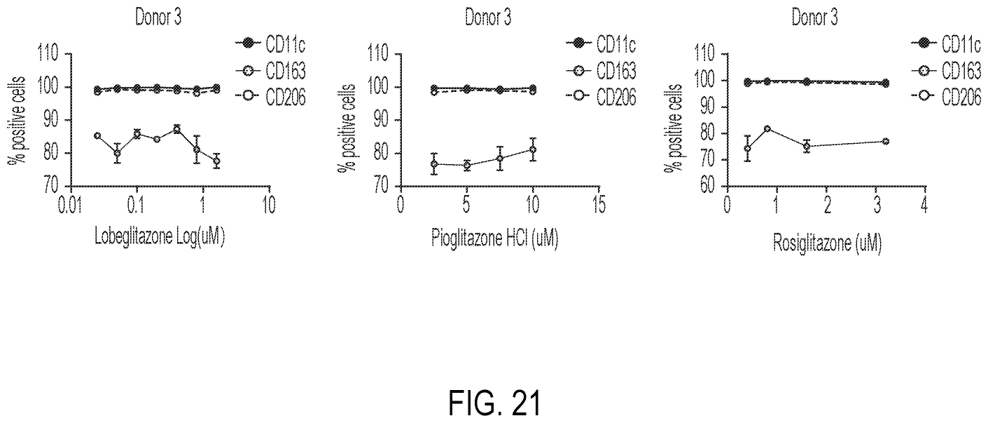

Detailed cell phenotype was presented in FIGS. 19, 20 and 21.

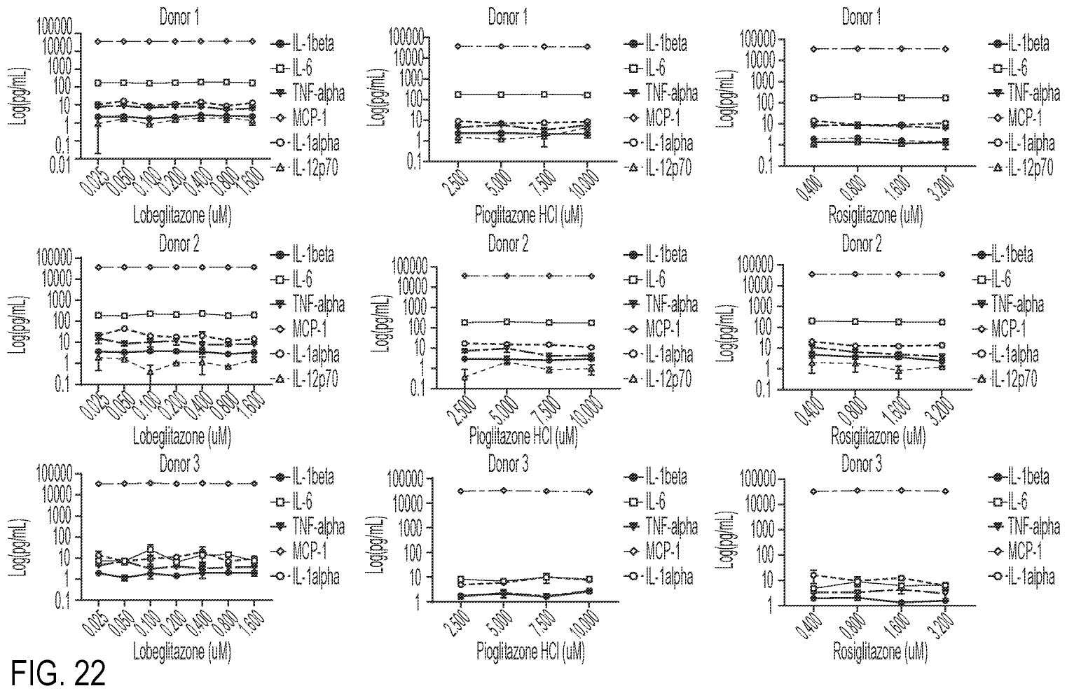

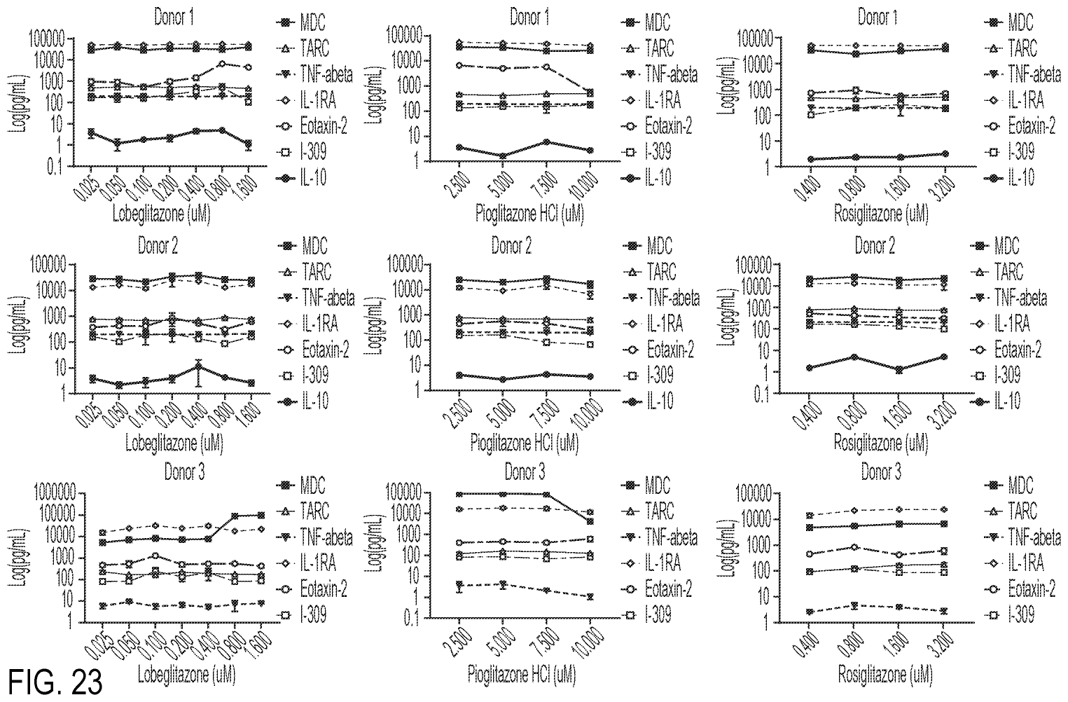

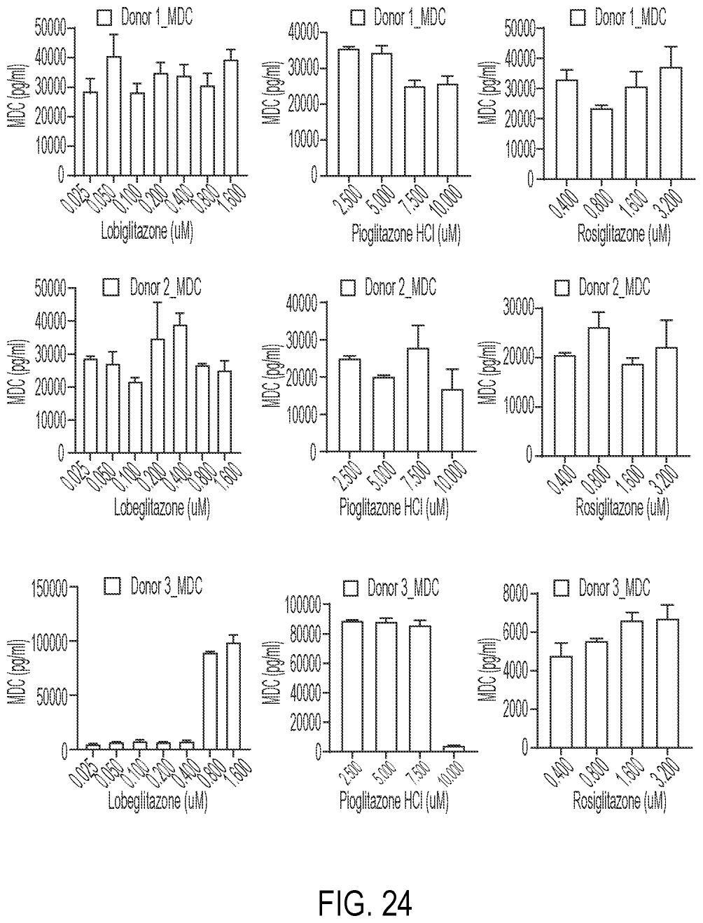

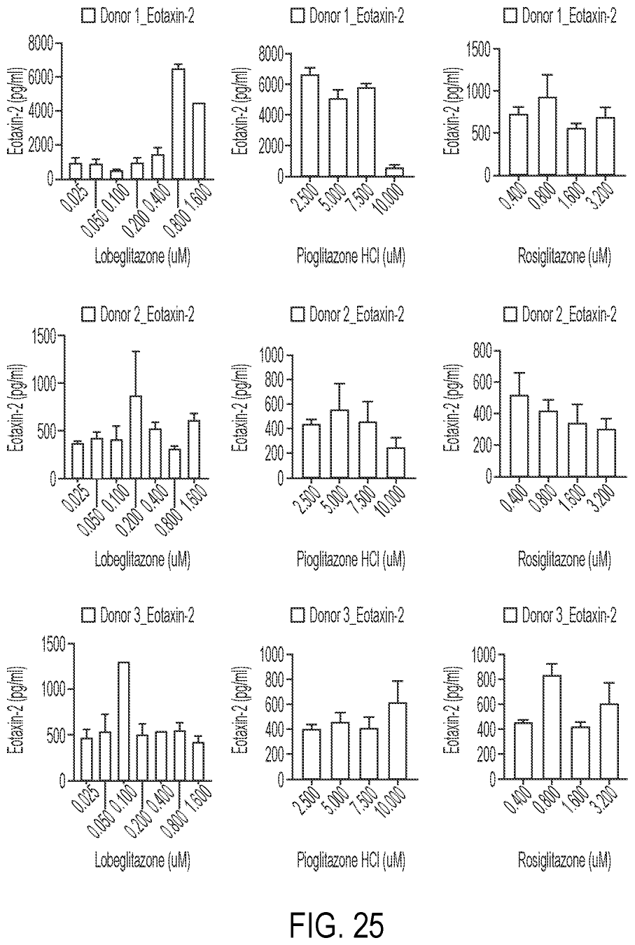

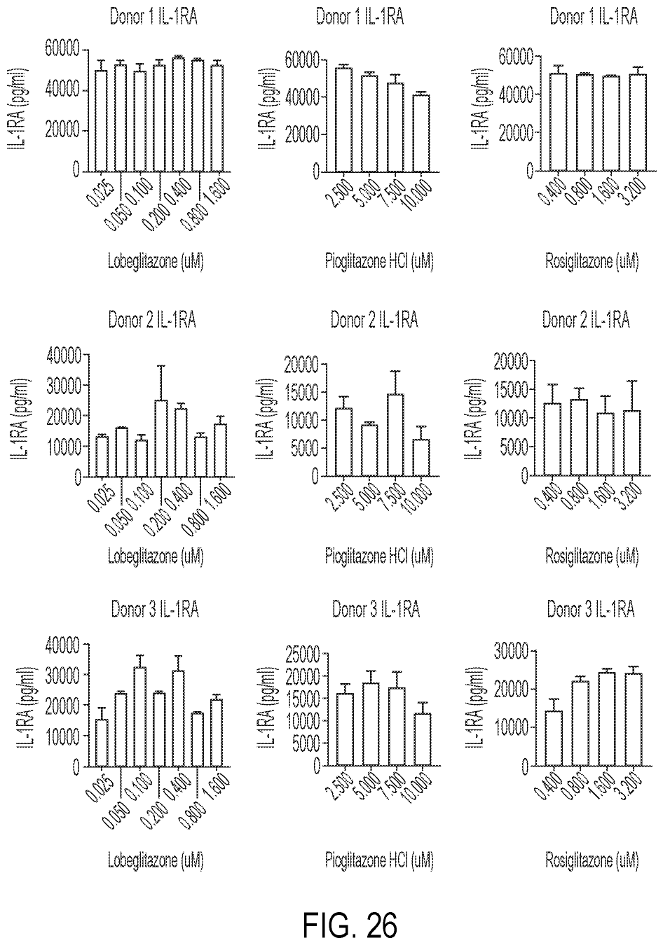

Cytokine and chemokines expression were quantified using MSD. FIGS. 22 and 23 showed overall M1 and M2 associated cytokines expression, respectively. Detailed M2 associated cytokines MDC, Eotaxin-2, and IL-1RA are presented in FIGS. 24, 25 and 26.

### CONCLUSION

This study evaluated the effects of lobeglitazone, pioglitazone, and rosiglitazone on M2 macrophage differentiation using flow cytometry and cytokine assays. As shown in the flow cytometry studies, M2 macrophage surface marker characterization showed that M107 promoted M2 repolarization in lower concentration (˜0.025 UM) compared to pioglitazone HCl (˜10 μM) and rosiglitazone (˜0.4 μM) among donor 1 and 2. In the cytokine analysis, M107 also showed a remarkable promotion of MDC expression (in donor 3) and Eotaxin-2 expression (in donor 1).

### Example 4: M1-to-M2 Macrophage Repolarization Assay

### Methodology

The purpose of this study was to evaluate the effects of lobeglitazone and other thiazolidinediones on M1-to-M2 macrophage repolarization. The test articles and vehicles used in this study were lobeglitazone sulfate, pioglitazone HCl, and rosiglitazone.

Media and solution reconstitution: (1) Prepare complete culture medium: add 50 mL of HI FBS (10%) and 5 mL (1%) of 100× Pen/Strep into 500 ml bottle of RPMI-1640; (2) Reconstitute LPS with sterilized water to 1 mg/ml stock concentration; (3) Reconstitute hIL-4 with sterilized water to 100 μg/ml stock concentration; (4) Reconstitute human M-CSF by solubilizing in sterilized water for 0.1 mg/ml by pipetting mix; (5) Reconstitute human GM-CSF by solubilizing in sterilized water for 0.1 mg/ml by pipetting mix; (6) Reconstitute test articles (M107, pioglitazone, rosiglitazone) with DMSO to reach 10 mM stock concentration.

M1-to-M2 macrophage repolarization assay timeline were as follows in Table 13.

TABLE 13

| Day | Action Items |
|---|---|
| Day 0 | Isolate monocytes from PBMC and differentiate in |
| | the presence of M-CSF into M2 macrophage |
| Day 4 | Add additional medium containing M-CSF |
| Day 6 | Stimulate the cells with LPS and IFN-γ for 2 days |
| Day 8 | Remove medium and replenish with cytokine free medium |
| | Add test compounds and incubate for 4 days |
| Day 12 | Add IL-4 and incubate for 2 days |
| Day 14 | Collect supernatant for cytokine and chemokines |
| | quantification |
| | Collect cells for flow |

Drug concentrations tested in the M1 differentiation assay are as in Table 14.

TABLE 14

| Drug | Testing concentration (μM) |
|---|---|
| Lobeglitazone sulfate | 0.025, 0.05, 0.1, 0.2, 0.4, 0.8, 1.6, 3.2 |
| Pioglitazone | 2.5, 5, 10 |
| Rosiglitazone | 0.2, 0.4, 0.8, 1.6, 3.2, 6.4 |

### Experimental Procedures (Monocyte Isolation, Treatment and Plating)

(1) Isolate monocytes from PBMC using StemCell® monocyte isolation kit.
(2) Count cell and adjust the cell density to 0.5×10⁶ cells/ml with fresh complete differentiation medium (add 10 μl M-CSF stock (100 μg/ml) 10 ml cell mixture to a final concentration of 50 ng/ml).
(3) Seed isolated monocyte 100 μL per well on 96-well plate.
(4) On day 4, add additional 100 μl medium containing M-CSF
(5) On day 6, add stimulator at 5× final concentration (200 μL medium in each well) to a final concentration of 50 ng/ml.
(6) On day 8, remove medium and replenish with 200 μL cytokine free medium, then add test compound (final concentration in μM) and incubate for 4 days.
(7) On day 12, add IL-4 and incubate for 2 days (add 50 μL of 100 ng/ml IL-4 to each well containing 200 μL medium to reach 20 ng/ml).
(8) On day 14, spin down the plates at 300×g for 5 min and collect the supernatant for cytokine quantification.
(9) M1 and M2 associated macrophage cytokines of interest are as follows in Table 15.

TABLE 15

| | M1/M2-associated cytokines |
|---|---|
| Cytokines | IL-1RA, IL-1a, IL-1b, IL-6, IL-10, IL-12p70, |
| | TNF-alpha, TNF-beta |
| Chemokines | CCL1 (1-309), CCL2 (MCP-1), CCL5 (RANTES; R-plex), |
| | CCL17 (TARC), CCL22 (MDC), CCL24 (eotaxin-2) |

The cells are then collected for flow analysis of viability/CD11c/CD163/CD206.

### Results

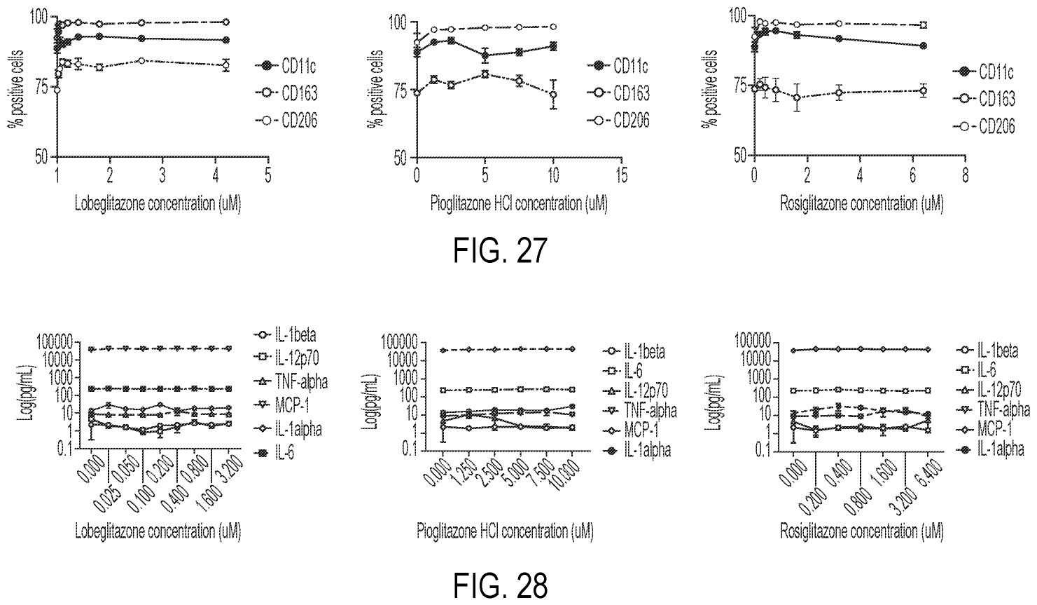

Cell surface marker CD163, CD11c, and CD206 were applied to characterize M2 repolarization via flow cytometry. Controls included No stain and staining with all surface marker antibodies to confirm the optimization of gating strategy. Detailed cell phenotype is presented in FIG. 27.

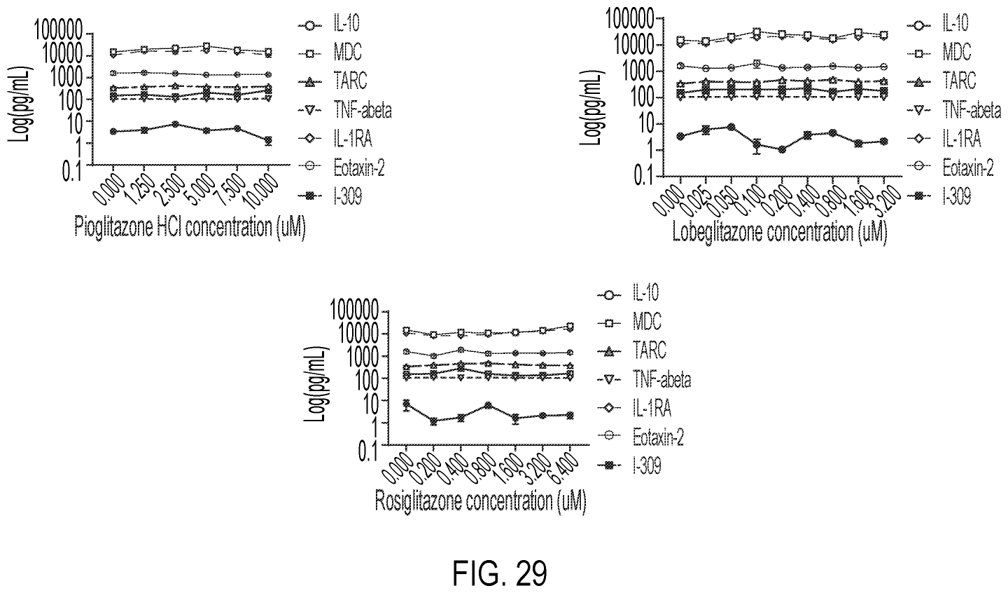

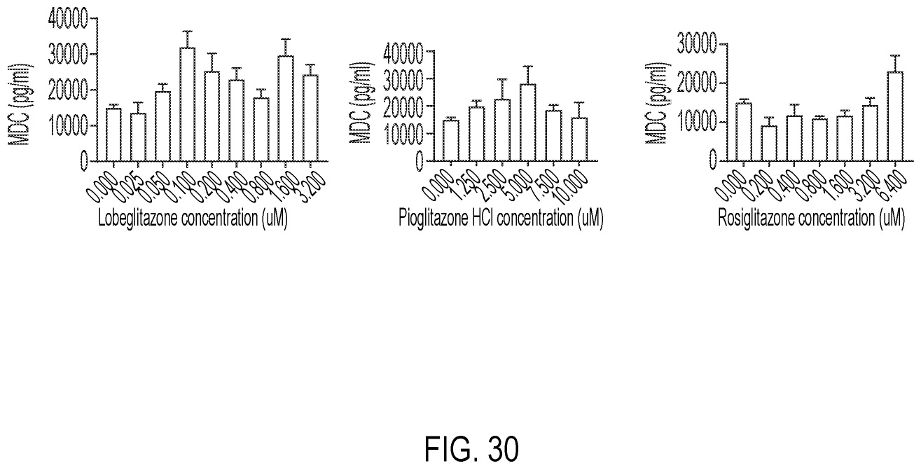

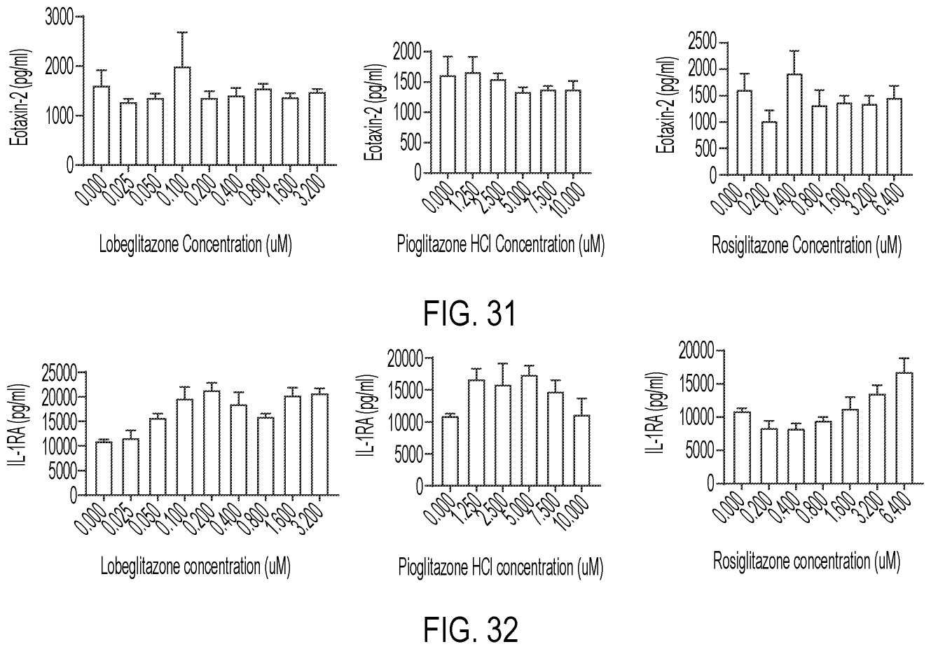

Cytokine and chemokines expression were quantified using MSD. FIGS. 28 and 29 showed overall M1 and M2 associated cytokine expression, respectively. Detailed M2 associated cytokines MDC, Eotaxin-2, and IL-1RA are presented in FIGS. 30-32.

### CONCLUSION

This study evaluated the effects of lobeglitazone, pioglitazone, and rosiglitazone on M1-to-M2 repolarization using flow cytometry and cytokine assays. As shown in the flow cytometry studies, M2 macrophage surface marker characterization showed that M107 promoted M1-to-M2 repolarization in a concentration as low as ˜0.05 μM. In the cytokine analysis, M107 also showed a remarkable promotion of MDC expression at 0.1 μM, as opposed to 5 μM and 6.4 μM for pioglitazone and rosiglitazone, respectively.

Unless otherwise indicated, all numbers expressing quantities of ingredients, properties such as molecular weight, reaction conditions, and so forth used in the specification and claims are to be understood as being modified in all instances by the term “about.” As u″ed he″ein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1.-13. (canceled)

14. A method of treating gastroparesis in a subject in need thereof, the method comprising administering to the subject a therapeutically effective amount of a pharmaceutical composition comprising lobeglitazone.

15.-17. (canceled)

18. The method of claim 14, wherein gastroparesis is idiopathic gastroparesis, diabetic gastroparesis, post-surgical gastroparesis, post-surgical gastroparesis, or medication-induced gastroparesis.

19.-20. (canceled)

21. The method of claim 14, wherein the subject is a human or a non-human mammal.

22. The method of claim 14, wherein the lobeglitazone is administered at a dose of 0.01 mg or higher.

23. The method of claim 14, wherein the lobeglitazone is administered at a dose between 0.01-5000 mg/day.

24. The method of claim 14, wherein the lobeglitazone is administered parenterally, enterally, topically, orally, sublingually, buccally, pulmonarily, intranasally, intravenously, intramuscularly, or subcutaneously.

25.-40. (canceled)

41. A method of inhibiting M1 macrophage differentiation, promoting M2 macrophage differentiation, or promoting M1-to-M2 macrophage repolarization in an individual, comprising administering an effective amount of lobeglitazone to the individual, optionally wherein as a result of the inhibition of M1 macrophage differentiation, a GI system disorder is treated in the individual, wherein the GI system disorder is selected from abdominal adhesions, acid reflux (GERD) in adults, acid reflux (GERD) in infants, anatomic problems of the lower GI tract, appendicitis, Barrett's esophagus, bowel control problems (fecal incontinence), celiac disease, colon polyps, constipation, Crohn's disease, cyclic vomiting syndrome, diarrhea, diverticulosis and diverticulitis, dumping syndrome, food poisoning, gallstones, gas, gastritis, gastroparesis, GI bleeding, hemorrhoids, indigestion (dyspepsia), inguinal hernia, intestinal pseudo-obstruction, IBD, IBS, lactose intolerance, liver disease, microscopic colitis, ostomy surgery of the bowel, pancreatitis, peptic ulcers (stomach ulcers), proctitis, short bowel syndrome, ulcerative colitis, viral gastroenteritis, or Zollinger-Ellison syndrome, or a condition associated therewith.

42.-49. (canceled)

50. A method of protecting the digestive system from damage, comprising contacting a cell of the gastrointestinal (GI) system with an effective amount of lobeglitazone.

51. The method of claim 50, wherein the cell is from mouth, pharynx (throat), esophagus, stomach, small intestine, large intestine, rectum, anus, salivary glands, liver, gallbladder, or pancreas.

52. The method of claim 50, wherein the GI system cell is an absorptive cell (enterocyte), a goblet cell, a pancreatic islet cell, an enteroendocrine cell, a hepatocyte, a paneth cell, a fenestrated hepatic endothelial cell, a kupffer cell, a serous cell, a gastric chief cell, a mucous cell, a smooth muscle cell, a gastric parietal cell, a myoepithelial cell, a stem cell, a gastric surface mucous cell, a pancreatic acinar cell, a taste bud, an interstitial cells of Cajal (ICC), or a neuronal cell.

53. The method of claim 50, wherein the cell is a human cell or a non-human mammalian cell.

54. The method of claim 50, wherein the cell is treated in vitro, ex vivo, or in vivo.

55. The method of claim 50, wherein the cell is in a subject having gastroparesis or is at risk of having gastroparesis.

56. The method of claim 55, wherein the gastroparesis is idiopathic gastroparesis, diabetic gastroparesis, post-surgical gastroparesis, or medication-induced gastroparesis.

57. The method of claim 14, further comprising a method selected from:
a method of alleviating signs and symptoms of gastroparesis, optionally wherein: the severity of signs and symptoms of gastroparesis are measured by the American Neurogastroenterology and Motility Society Gastroparesis Cardinal Symptom Index-Daily Diary (GCSI-DD), or the severity of signs and symptoms of gastroparesis are measured by the change of GCSI-DD score from baseline to 4-week treatment, 8-week treatment, 12-week treatment, or 1-year treatment;
a method of decreasing the severity of nausea in subjects with gastroparesis;
a method of decreasing the severity of early satiety in subjects with gastroparesis;
a method of decreasing the severity of postprandial fullness in subjects with gastroparesis;
a method of decreasing the severity of upper abdominal pain in subjects with gastroparesis;
a method of decreasing the severity or number of vomiting episodes in subjects with gastroparesis;
a method of decreasing the overall severity of gastroparesis;
a method of decreasing the GI inflammation, as indicated by any one or more decrease of TNF-α, IL-10, CCL2, and IL-1β gene expression in cells of a subject experiencing GI inflammation; or
a method of slowing down, stopping, or reversing the progress of gastroparesis.
